(19) United States
(12) Patent Application Publication
(10) Pub. No.: US 2007/0292439 A1
(43) Pub. Date: Dec. 20, 2007

(54) Antagonist Anti-Cd40 Monoclonal Antibodies and Methods for Their Use

(75) Inventor: Mohammad Luqman, Emeryville, CA (US)

Correspondence Address: NOVARTIS VACCINES AND DIAGNOSTICS INC., INTELLECTUAL PROPERTY R338, P.O. BOX 8097, Emeryville, CA, 94662-8097, US

(73) Assignee: Novartis Vaccines and Diagnostics, Inc., Emeryville, CA (US)

(21) Appl. No.: 11/587,911

(22) PCT Filed: Apr. 27, 2005
(86) PCT No.: PCT/US05/14359

§ 371 Date: Jul. 27, 2007

Related U.S. Application Data
(60) Provisional application No. 60/565,775, filed on Apr. 27, 2004.

(51) Int. Cl. A61K 39/395; A61P 37/00; C07K 16/00; C12N 5/00; C12P 21/08; G01N 33/574

(52) U.S. Cl. 424/172.1; 435/346; 435/375; 436/501; 530/387.1; 530/387.3; 530/388.15; 530/388.2

(57) ABSTRACT

Compositions and methods for inhibiting CD40-directed activities that are mediated via the binding of C4BP to CD40 are provided. The compositions of the invention include anti-CD40 antibodies, or antigen-binding fragments thereof, that have the following characteristics: 1) are free of significant CD40 agonist activity when bound to CD40 antigen; and 2) are capable of specifically binding to CD40 antigen expressed on the surface of cells, wherein this binding to CD40 antigen blocks C4BP-mediated CD40 signaling, thereby inhibiting one or more CD40-directed activities. These antagonist anti-CD40 antibodies can effectively be used to treat CD40-associated diseases that are mediated by C4BP stimulation of CD40 signaling, including cancers, such as B cell-related cancers and solid tumors, and diseases or disorders that have an autoimmune and/or inflammatory component, including organ and tissue transplant rejection.

### FIELD OF THE INVENTION

This invention relates to antibodies capable of binding to the CD40 cell surface antigen, thereby blocking C4BP-mediated CD40 signaling, and methods of using the antibodies, including methods for treatment of diseases mediated by C4BP stimulation of CD40 signaling on CD40-expressing cells.

### BACKGROUND OF THE INVENTION

CD40 is a cell-surface antigen that is related to the human nerve growth factor (NGF) receptor, tumor necrosis factor-α (TNF-α) receptor, and Fas. Expression of this member of the TNF receptor family was first characterized on B lymphocytes, but recent findings show that it is broadly expressed on a number of cell types. In the hematopoietic system, CD40-expressing cells include CD34+ hematopoietic progenitors, B cell progenitors, mature B lymphocytes (both normal and malignant), plasma cells, monocytes, dendritic cells, eisonophils, basophils, and some T lymphocytes. Non-hematopoietic CD40-expressing cells include endothelial cells, epithelial cells, and fibroblasts. CD40 expression also occurs on synovial membranes in rheumatoid arthritis, activated platelets, inflamed vascular smooth muscle cells, and dermal fibroblasts. It is expressed at low levels on vascular endothelial cells and is up-regulated in areas of local inflammation. Given its broad expression pattern, CD40 likely plays a more general role in immune regulation by mediating interactions between T-cells and B-cells as well as other cell types. See, for example, Kooten and Banchereau (1997) Frontiers in Biosciences 2:1-11.

The effect of activation of CD40 signaling mediated by binding of its ligand, CD40L or CD154, to the CD40 receptor depends upon the cell type involved. Thus, activation of CD40 signaling on B cells stimulates B cell proliferation and differentiation, antibody production, isotype switching, and B cell memory generation, while activation of CD40 signaling on dendritic cells and monocytes leads to expression of costimulatory molecules and secretion of inflammatory cytokines (e.g., IL-8, IL-12, and TNF-alpha), providing for efficient activation of T lymphocytes. Both agonist anti-CD40 monoclonal antibodies and oligomeric soluble CD40L can stimulate the CD40 signaling pathway (see, for example, International Publication WO 00/75348 and U.S. Pat. No. 6,087,329). The overall effect of these signals is to markedly enhance T-cell priming and, for CD40-expressing dendritic cells, to favor the generation of Th1 cellular immune responses.

The complement cascade plays a critical role in in vivo innate immune responses. See, for example, William Paul (1993) Fundamental Immunology (3^{rd ed., Raven Press), pp.} 924-929. During the complement cascade, antigen-bound IgG initiates a series of hydrolytic reactions that ultimately creates a membrane attack complex (MAC) that punctures bacterial cell membranes. C4 is a critical component in this pathway. C4, which is normally inactive in the serum, can be cleaved by C1 that is present in the antigen-antibody complex. C1 cleaves C4 into the C4a and C4b fragments. C4a is a small soluble protein that acts as a weak anaphylotoxin. C4b binds the surface of the bacterial cell membrane and can either act independently as an opsonin, or it can bind C3b to form the C3 convertase of the classical complement fixation pathway. The generation of target bound C4b is an inefficient process and only 5%-10% of C4b becomes substrate bound. Serum regulatory proteins are responsible for clearing the excess C4b.

C4b binding protein (C4BP) is one of the of the serum regulatory proteins that binds C4b. Synthesized by liver cells and activated monocytes, it is a co-factor in the Factor-I-mediated catabolism of C4b and C3b. See, for example, Dahlback and Hildebrand (1983) Biochem. J. 209:857; Lappin and Whaley (1990) Biochem. J. 271:767; Kusada-Funakoshi et al. (1991) Biochem. Med. Metab. Biol. 45:350; Blom et al. (2001) J. Biol. Chem. 276:27136; Blom et al. (2001) J. Immunol. 166:6764; Blom et al. (2003) Mol. Immunol. 399:547. In circulation, C4BP is present in three isoforms that are based on differing combinations of the alpha (70kDa) and beta (45 kDa) chains. The predominant isoform is an octamer of 7 alpha subunits and 1 beta subunit α7β1) (Pardo-Manuel et al. (1990) Proc. Natl. Acad. Sci. USA 87:4529; Kask et al. (2002) Biochemistry 41:9348). Reportedly, C4BP is also capable of binding CD40, and in vitro binding to CD40 on B cells can activate these cells in a manner that mimics CD40L binding (Brodeur et al. (2003) Immunity 18:837). Moreover, this in vitro activity is correlated with the ability of C4BP to bind the CD40 protein directly in a region of the molecule that does not competitively inhibit binding for the CD40 ligand. CD40 stimulation is a key mediator for autoimmune diseases. C4BP may directly induce CD40 signaling, and thereby contribute to initiation and progression of autoimmune and inflammatory diseases. C4BP can also additively or synergistically work with CD40 ligand to exacerbate CD40 stimulation and contribute to disease initiation and progression. Thus, binding of C4BP to the C4BP binding site on CD40 may transduce aberrant or undesirable signals to cells expressing CD40.

Blocking of CD40 engagement and activation has the potential to suppress antibody and cell-mediated immune responses. Anti-CD40 antagonist antibodies could be used to treat autoimmune disorders such as systemic lupus, psoriasis, multiple sclerosis, inflammatory bowel disease, and rheumatoid arthritis. Such antibodies could also be used to prevent rejection of organ and tissue grafts by suppressing autoimmune response, to treat lymphomas by depriving malignant B lymphocytes of the activating signal provided by CD40, and to deliver toxins to CD40-bearing cells in a specific manner. Two types of anti-CD40 antagonist monoclonal antibodies can block CD40 activation: 1) those that block CD40 ligand-mediated CD40 signaling, and 2) those that block C4BP-mediated CD40 signaling.

Given the putative role of C4BP in the CD40 activation pathway, there is a need for interfering with C4BP-mediated CD40 signaling so that diseases associated with C4BP-CD40 binding interaction can be treated.

### BRIEF SUMMARY OF THE INVENTION

Compositions and methods for inhibiting CD40-directed activities that are mediated via the binding of C4BP to CD40 are provided, as are methods for treating CD40-associated diseases that are mediated via this C4BP-CD40 binding interaction. The compositions of the invention include anti-CD40 antibodies, or antigen-binding fragments thereof, that have the following characteristics: 1) are free of significant CD40 agonist activity when bound to CD40 antigen on CD40-expressing cells; and 2) are capable of specifically binding to CD40 antigen expressed on the surface of cells, wherein this binding to CD40 antigen blocks C4BP-mediated CD40 signaling of these cells, thereby inhibiting one or more CD40-directed activities. These anti-CD40 antibodies or antigen-binding fragments thereof exhibit a strong single-site binding affinity for the CD40 cell-surface antigen. In some embodiments, the antibodies of the invention exhibit a dissociation equilibrium constant (K_{D}) for CD40 of at least 10⁻⁵ M, at least 3×10⁻⁵ M, preferably at least 10⁻⁶ M to 10⁻⁷ M, more preferably at least 10⁻⁸ M to about 10⁻¹² M. Suitable monoclonal antibodies have human constant regions; preferably they also have wholly or partially humanized framework regions; and most preferably are fully human antibodies or antigen-binding fragments thereof. Compositions also include hybridoma cell lines producing these antibodies or antigen-binding fragments thereof, and pharmaceutical compositions comprising these antibodies or antigen-binding fragments thereof.

Methods for inhibiting a CD40-directed activity of a CD40-expressing cell comprise contacting the cell with an effective amount of an anti-CD40 antibody of the invention, or an effective amount of an antigen-binding fragment thereof. CD40-directed activities that can be inhibited include, but are not limited to, cell growth and proliferation, cell differentiation, antibody production, cell memory generation, isotype switching, intercellular adhesion, secretion of cytokines, secretion of metalloproteases, and expression of cell adhesion molecules. The anti-CD40 antibodies of the invention can be used to treat CD40-associated diseases that are mediated via C4BP stimulation of a CD40-directed activity, including, but not limited to, cancers, including B cell-related cancers and solid tumors, and diseases or disorders having an autoimmune and/or inflammatory component, including organ and tissue transplant rejections. Methods for identifying antibodies that have antagonist activity toward CD40 and that interfere with C4BP-mediated CD40 signaling in CD40-expressing cells are also provided.

### DETAILED DESCRIPTION OF THE INVENTION

The present invention is directed to anti-CD40 antibodies and methods for their use. These anti-CD40 antibodies, and antigen-binding fragments thereof, are capable of specifically binding to a CD40 antigen expressed on the surface of a cell, and are free of significant CD40 agonist activity when bound to CD40 antigen. Binding of these anti-CD40 antibodies, or antigen binding fragments thereof, to CD40 antigen on CD40-expressing cells effectively blocks C4b binding protein (C4BP)-mediated CD40 signaling of these cells. By “C4BP-mediated CD40 signaling” is intended the stimulation of CD40 signaling that occurs when C4BP binds to CD40 antigen expressed on the surface of a cell. By “blocks” or “blocking” is intended the partial or complete inhibition of the CD40 signaling that would normally result from the binding of C4BP to its binding site on CD40 expressed on the surface of a cell in the absence of an antagonist such as the anti-CD40 antibodies of the present invention. Blocking of this CD40 signaling provides a means for inhibiting, either partially (i.e., reduction in an activity) or completely (i.e., prevention of an activity), one or more CD40-directed activities that results from C4BP-mediated CD40 signaling. Inhibition of these CD40-directed activities can advantageously be used for treating CD40-associated diseases, including, but not limited to, cancers, such as B cell-related cancers and solid tumors, and diseases or disorders that have an autoimmune and/or inflammatory component, including organ and tissue transplant rejection, as noted herein below.

The following terms appear throughout the invention disclosure and are further defined herein below.

“Tumor,” as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include, but are not limited to, lymphoma and leukemia, and solid tumors.

“Antibodies” and “immunoglobulins” (Igs) are glycoproteins having the same structural characteristics. While antibodies exhibit binding specificity to an antigen, immunoglobulins include both antibodies and other antibody-like molecules that lack antigen specificity. Polypeptides of the latter kind are, for example, produced at low levels by the lymph system and at increased levels by myelomas.

The term “antibody” is used in the broadest sense and covers fully assembled antibodies, antibody fragments that can bind antigen (e.g., Fab, F(ab′)₂, Fv, single chain antibodies, diabodies), and recombinant peptides comprising the foregoing.

The term “monoclonal antibody” as used herein refers to an antibody obtained from a population of substantially homogeneous antibodies, i.e., the individual antibodies comprising the population are identical except for possible naturally occurring mutations that may be present in minor amounts.

“Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity determining regions (CDRs) or hypervariable regions both in the light chain and the heavy-chain variable domains. The more highly conserved portions of variable domains are termed the framework (FR) regions. The variable domains of native heavy and light chains each comprise four FR regions, largely adopting a β-pleated sheet configuration, connected by three CDRs, which form loops connecting, and in some cases forming part of, the β-pleated sheet. The CDRs in each chain are held together in close proximity by the FR regions and, with the CDRs from the other chain, contribute to the formation of the antigen-binding site of antibodies (see Kabat et al. (1991) NIH Publ. No. 91-3242, Vol. I, pages 647-669). The constant domains are not involved directly in binding an antibody to an antigen but exhibit various effector functions, such as Fc receptor (FcR) binding, participation of the antibody in antibody-dependent cellular toxicity, initiation of complement dependent cytotoxicity, and mast cell degranulation.

The term “hypervariable region” refers to the amino acid residues of an antibody that are responsible for antigen binding. The hypervariable region comprises amino acid residues from a complementarity determining region (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest (5th ed.; Public Health Service, National Institute of Health, Bethesda, Md.) and/or those residues from a “hypervariable loop” (i.e., residues 26-32 (L1), 50-52 (L2), and 91-96 (L3) in the light-chain variable domain and 26-32 (H1), 53-55 (H2), and 96-101 (H3) in the heavy-chain variable domain; Clothia and Lesk (1987) J. Mol. Biol. 196:901). “Framework” or “FR” residues are those variable domain residues other than the hypervariable region residues.

“Antibody fragments” comprise a portion of an intact antibody, preferably the antigen-binding or variable region of the intact antibody. Examples of antibody fragments include Fab, Fab′, F(ab′)2, and Fv fragments; diabodies; linear antibodies (Zapata et al. (1995) Protein Eng. 10: 1057); single-chain antibody molecules; and multispecific antibodies formed from antibody fragments. Papain digestion of antibodies produces two identical antigen-binding fragments, termed “Fab” fragments, each with a single antigen-binding site and a residual “Fc” fragment, whose name reflects its ability to crystallize readily. Pepsin treatment yields an F(ab′)2 fragment that has two antigen-combining sites capable of cross-linking antigen.

“Fv” is the minimum antibody fragment that contains a complete antigen recognition and binding site. In a two-chain Fv species, this region consists of a dimer of one heavy- and one light-chain variable domain in tight, non-covalent association. In a single-chain Fv species, one heavy- and one light-chain variable domain can be covalently linked by flexible peptide linker such that the light and heavy chains can associate in a “dimeric” structure analogous to that in a two-chain Fv species. It is in this configuration that the three CDRs of each variable domain interact to define an antigen-binding site on the surface of the V_{H}—V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H}1) of the heavy chain. Fab fragments differ from Fab′ fragments by the addition of a few residues at the carboxy terminus of the heavy-chain C_{H}1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two classes, called kappa (κ) and lambda (λ), based on the amino acid sequences of their constant domains.

Depending on the amino acid sequence of the constant domain of the heavy chains, immunoglobulins comprise different classes. There are five major classes of human immunoglobulins: IgA, IgD, IgE, IgG, and IgM. In humans, these classes are further divided into subclasses (isotypes), e.g., IgG1, IgG2, IgG3, IgG4, IgA1, and IgA2. The heavy-chain constant domains that correspond to the different classes of immunoglobulins are termed alpha, delta, epsilon, gamma, and mu, respectively. The subunit structures and three-dimensional configurations of different classes of immunoglobulins are well known. Different isotypes have different effector functions. For example, human IgG1 and IgG3 isotypes have antibody-dependent cell-mediated cytotoxicity (ADCC) activity.

The word “label” when used herein refers to a compound or composition that is conjugated directly or indirectly to the antibody so as to generate a “labeled” antibody. The label may be detectable by itself (e.g., radioisotope labels or fluorescent labels) or, in the case of an enzymatic label, may catalyze chemical alteration of a substrate compound or composition that is detectable. Radionuclides that can serve as detectable labels include, for example, I-131, I-123, I-125, Y-90, Re-188, Re-186, At-211, Cu-67, Bi-212, and Pd-109. The label might also be a non-detectable entity such as a toxin.

The term “antagonist” is used in the broadest sense, and includes any molecule that partially or fully blocks, inhibits, or neutralizes a biological activity of a target protein molecule disclosed herein or the transcription or translation thereof. Antagonist antibodies are antibodies that bind a cell-associated antigen without transducing a signal to the cell. A signal may include any biochemical reaction that results in a change in the cell's state including inducing proliferation and/or survival, inducing apoptosis, inducing phosphorylation of other proteins, inducing release of calcium stores, and inducing cytokine secretion.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 amino acid residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG), and Pluronics. Administration “in combination with” one or more further therapeutic agents includes simultaneous (concurrent) and consecutive administration in any order.

A “host cell,” as used herein, refers to a microorganism or a eukaryotic cell or cell line cultured as a unicellular entity that can be, or has been, used as a recipient for a recombinant vector or other transfer polynucleotides, and include the progeny of the original cell that has been transfected. It is understood that the progeny of a single cell may not necessarily be completely identical in morphology or in genomic or total DNA complement as the original parent, due to natural, accidental, or deliberate mutation.

“Human effector cells” are leukocytes that express one or more FcRs and perform effector functions. Preferably, the cells express at least FcγRIII and carry out antibody-dependent cell-mediated cyotoxicity (ADCC) effector function. Examples of human leukocytes that mediate ADCC include peripheral blood mononuclear cells (PBMC), natural killer (NK) cells, monocytes, macrophages, eosinophils, and neutrophils, with PBMCs and NK cells being preferred. Antibodies that have ADCC activity are typically of the IgG1 or IgG3 isotype. In addition to isolating IgG1 and IgG3 antibodies, ADCC-mediating antibodies can be synthesized by engineering a variable region from a non-ADCC antibody or variable region fragment to an IgG1 or IgG3 isotype constant region.

The terms “Fc receptor” or “FcR” are used to describe a receptor that binds to the Fc region of an antibody. The preferred FcR is a native-sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see Daeron (1997) Annu. Rev. Immunol. 15:203). FcRs are reviewed in Ravetch and Kinet (1991) Annu. Rev. Immunol 9:457; Capel et al. (1994) Immunomethods 4:25; and de Haas et al. (1995) J. Lab. Clin. Med. 126:330. Other FcRs, including those to be identified in the future, are encompassed by the term “FcR” herein. The term also includes the neonatal receptor, FcRn, which is responsible for the transfer of maternal IgGs to the fetus (Guyer et al. (1976) J. Immunol. 117:587, and Kim et al. (1994) J. Immunol. 24:249).

There are a number of methods for synthesizing human antibodies. For example, secreting cells can be immortalized by infection with the Epstein-Barr virus (EBV). However, EBV-infected cells are difficult to clone and usually produce only relatively low yields of immunoglobulin (James and Bell (1987) J. Immunol. Methods 100:5). Eventually, the immortalization of human B cells may be achieved by introducing a defined combination of transforming genes. Such a possibility is highlighted by a recent demonstration that the expression of the telomerase catalytic subunit together with the SV40 large oncoprotein and an oncogenic allele of H-ras resulted in the tumorigenic conversion of normal human epithelial and fibroblast cells (Hahn et al. (1999) Nature 400:464). Transgenic animals (e.g., mice), upon immunization, can be capable of producing a repertoire of human antibodies in the absence of endogenous immunoglobulin production (Jakobovits et al. (1993) Nature 362:255; Lonberg and Huszar (1995) Int. Rev. Immunol. 13:65; Fishwild et al. (1996) Nat. Biotechnol. 14:845; Mendez et al. (1997) Nat. Genet. 15:146; Green et al. (1999) J. Immunol. Methods 231:11; Tomizuka et al. (2000) Proc. Natl. Acad. Sci. USA 97:722-727; reviewed in Little et al. (2000) Immunol. Today 21: 364). For example, it has been described that the homozygous deletion of the antibody heavy chain joining region (J_{H}) gene in chimeric and germ-line mutant mice results in complete inhibition of endogenous antibody production (Jakobovits et al. (1993) Proc. Natl. Acad. Sci. USA 90:2551). Transfer of the human germ-line immunoglobulin gene array in such germ-line mutant mice results in the production of human antibodies upon antigen challenge (Jakobovits et al. (1993) Nature 362:255). Mendez et al. (1997) (Nature Genetics 15:146) have generated a line of transgenic mice that, when challenged with an antigen, generates high affinity fully human antibodies. This was achieved by germ-line integration of megabase human-heavy chain and light-chain loci into mice with deletion into endogenous J_{H} segment as described above. These mice (XenoMouse® II technology (Abgenix; Fremont, Calif.)) harbor 1,020 kb of human heavy-chain locus containing approximately 66 V_{H} genes, complete D_{H} and J_{H} regions, and three different constant regions, and also 800 kb of human κ locus containing 32 Vκ genes, Jκ segments, and Cκ genes. The antibodies produced in these mice closely resemble that seen in humans in all respects, including gene rearrangement, assembly, and repertoire. The human antibodies are preferentially expressed over endogenous antibodies due to deletion in endogenous segment that prevents gene rearrangement in the murine locus. Such mice may be immunized with an antigen of particular interest.

Sera from such immunized animals may be screened for antibody reactivity against the antigen of interest. Lymphocytes may be isolated from lymph nodes or spleen cells and may further be selected for B cells by selecting for CD138-negative and CD19-positive cells. In one aspect, such B cell cultures (BCCs) may be fused to myeloma cells to generate hybridomas as detailed above.

In another aspect, such B cell cultures may be screened further for reactivity against the initial antigen, preferably. Such screening includes ELISA with the target/antigen protein, a competition assay with known antibodies that bind the antigen of interest, and in vitro binding to transiently transfected CHO or other cells that express the target antigen.

The anti-CD40 antibodies of the invention and methods for their use are described in more detail below.

Antagonist Anti-CD40 Antibodies

The anti-CD40 antibodies of the present invention, and antigen-binding fragments thereof, preferably have antagonist activity on CD40, and hence are referred to herein as “antagonist” anti-CD40 antibodies. More specifically, the antagonist anti-CD40 antibodies of the invention specifically bind to CD40 antigen on the surface of CD40-expressing cells, whereby this binding blocks C4BP-mediated CD40 signaling. Blocking of this signaling process results in inhibition of one or more CD40-directed activities that are initiated when an agonist, such as C4BP, binds to the CD40 cell surface antigen.

By “CD40 antigen,” “CD40 cell surface antigen,” “CD40 receptor,” or “CD40” is intended a transmembrane glycoprotein that belongs to the tumor necrosis factor (TNF) receptor family (see, for example, U.S. Pat. Nos. 5,674,492 and 4,708,871; Stamenkovic et al. (1989) EMBO 8:1403; Clark (1990) Tissue Antigens 36:33; Barclay et al. (1997) The Leucocyte Antigen Facts Book (2d ed.; Academic Press, San Diego)). Two isoforms of human CD40, encoded by alternatively spliced transcript variants of this gene, have been identified. The first isoform (also known as the “long isoform” or “isoform 1”) is expressed as a 277-amino-acid precursor polypeptide (first reported as GenBank Accession No. CAA43045, and identified as isoform 1 in GenBank Accession No. NP_{—}001241; encoded by GenBank Accession Nos. X60592 and NM_{—}001250)), which has a signal sequence represented by the first 19 residues. The second isoform (also known as the “short isoform” or “isoform 2”) is expressed as a 203-amino-acid precursor polypeptide (GenBank Accession No. NP_{—}690593; encoded by GenBank Accession No. NM_{—}152854), which also has a signal sequence represented by the first 19 residues. The precursor polypeptides of these two isoforms of human CD40 share in common their first 165 residues. The precursor polypeptide of the short isoform is encoded by a transcript variant that lacks a coding segment, which leads to a translation frame shift; the resulting CD40 isoform contains a shorter and distinct C-terminus (residues 166-203 of GenBank Accession No. NP_{—}690593) from that contained in the long isoform of CD40 (C-terminus shown in residues 166-277 of GenBank Accession No. CAA43045 and GenBank Accession No. NP_{—}001241). For purposes of the present invention, the term “CD40 antigen,” “CD40 cell surface antigen,” “CD40 receptor,” or “CD40” encompasses both the short and long isoforms of CD40.

The CD40 receptor is displayed on the surface of a variety of cell types, as described elsewhere herein. By “displayed on the surface” and “expressed on the surface” is intended that all or a portion of the CD40 antigen is exposed to the exterior of the cell. The displayed or expressed CD40 antigen may be fully or partially glycosylated.

By “C4b binding protein” or “C4BP” is intended a soluble peptide or any fragment thereof including at least a portion of an alpha subunit (GenBank Accession No. NP_{—}000706, encoded by GenBank Accession No. NM_{—}000715; Kask et al. (2002) Biochemistry 41:9349) or a portion of a beta subunit (GenBank Accession No. NP_{—}000707, encoded by GenBank Accession No. NM_{—}000716; Webb et al. (2003) Eur. J. Biochem. 270:93). The term “C4BP” as used herein may include individual alpha or beta subunits or larger heteromers comprising these subunits such as the three serum isoforms: α7β1 (the predominant isoform in serum), α7β0, and α6β1. By the “C4BP binding site on CD40” is intended the region of the CD40 antigen where any portion of any C4BP subunit binds. See, for example, Brodeur et al. (2003) Immunity 18:837, herein incorporated by reference in its entirety. The binding site may comprise a linear determinant on CD40 or it may comprise a binding domain formed by discontiguous amino acids that form a C4BP binding site via secondary or tertiary conformation, or a combination thereof. In some instances, the C4BP binding site interacts exclusively with a C4BP alpha subunit.

When C4BP binds to the CD40 antigen on CD40-expressing cells, it serves as an agonist of CD40 signaling, and is thus said to have CD40 agonist activity. By “agonist activity” is intended that the substance functions as an agonist. An agonist combines with a receptor on a cell and initiates a reaction or activity that is similar to or the same as that initiated by the receptor's natural ligand, for example, CD40L in the case of CD40. An agonist of CD40, such as C4BP, induces any or all of, but not limited to, the following activities that occur when CD40 transduces a signal: proliferation and differentiation of antigen presenting cells (APCs); B cell antibody production; intercellular adhesion; B cell memory generation; B cell isotype switching (especially IgE isotype switching in the presence of IL-4); upregulation of cell-surface expression of MHC Class II, CD54, CD95, and CD80/86 in APCs; upregulation of bcl-x_{L}, A20, diacylglycerol kinase α, p38, and c-myc gene expression; nuclear translocation of NFκB; secretion of cytokines such as IL-2, IL-4, IL-5, IL-6, IL-8, IL-10, IL-12, and TNFα; secretion of metalloproteases such as MMP-I/collagenase and MMP-9/gelatinase B; and expression of cell adhesion molecules such as E-selectin, VCAM-1, and ICAM-1. For purposes of the present invention, such activities are referred to as “CD40-directed activities,” and the induction of such activities by the binding of C4BP to its binding site on CD40 is referred to as “C4BP-mediated CD40 signaling.”

The anti-CD40 antibodies of the invention, and antigen-binding fragments thereof, have antagonist activity with respect to their binding interaction with CD40. This antagonist activity results from the anti-CD40 antibodies ability to block C4BP-mediated CD40 signaling when these antibodies bind to CD40 or to send a negative signal through CD40. By “antagonist activity” is intended that the substance functions as an antagonist. An antagonist anti-CD40 antibody of the invention prevents or reduces induction of any one or more of the CD40-directed activities induced by binding of the CD40 receptor to an agonist ligand, particularly C4BP. The antagonist anti-CD40 antibodies of the invention may reduce induction of any one or more of the CD40-directed activities induced by the binding of C4BP to CD40 by 5%, 10%, 15%, 20%, 25%, 30%, 35%, preferably 40%, 45%, 50%, 55%, 60%, more preferably 70%, 80%, 85%, and most preferably 90%, 95%, 99%, or 100%. Methods for measuring anti-CD40 antibody and C4BP binding specificity and antagonist activity are known to one of skill in the art and include, but are not limited to, standard competitive binding assays, assays for dendritic cell induction of T cell proliferation, assays for monitoring immunoglobulin secretion by B cells, B cell proliferation assays, Banchereau-Like-B cell proliferation assays, T cell helper assays for antibody production, co-stimulation of B cell proliferation assays, and assays for upregulation of APC activation markers. See, for example, such assays disclosed in Brodeur et al. (2003) Immunity 18:837, WO 00/75348, and U.S. Pat. No. 6,087,329, herein incorporated by reference. In some embodiments, binding to CD40 displayed on the surface of human cells blocks C4BP-mediated CD40 signaling, resulting in inhibition of proliferation and differentiation of these human cells. Thus, the antagonist anti-CD40 antibodies of the invention include those antibodies that can exhibit antagonist activity toward normal and neoplastic human cells expressing the cell-surface CD40 antigen.

The antagonist activity of the anti-CD40 antibodies of the present invention is manifested via the blocking of C4BP-mediated CD40 signaling, for example, by competitive or steric interference with the binding of C4BP to its binding site on CD40. By “competitively inhibit” is intended that the anti-CD40 antibody, or antigen-binding fragment thereof, binds the same C4BP binding site on CD40, or at least a portion thereof, as native soluble C4BP, thereby inhibiting the binding of C4BP to its binding site on CD40. By “sterically inhibit” or “steric inhibition” is intended that the anti-CD40 antibody, or antigen-binding fragment thereof, binds outside, or at least partially outside, the C4BP binding site on CD40, wherein the antibody still inhibits C4BP or fragment thereof from binding to CD40 by steric interference or disruption of the structure of the C4BP binding site on CD40, or some combination thereof. In some embodiments, binding of the antagonist anti-CD40 antibody or antigen-binding fragment thereof to the CD40 antigen prevents CD40 signal transduction when C4BP ligates to CD40 antigen. One of skill could determine whether an antibody competitively or sterically interferes with the binding of C4BP to CD40, or prevents CD40 signal transduction with the binding of C4BP to its binding site on CD40, using standard methods well known in the art.

When the antagonist anti-CD40 antibodies of the invention bind CD40 displayed on the surface of CD40-expressing cells, such as normal and neoplastic human B cells, and human dendritic cells, the antibodies are free of significant agonist activity when bound to CD40. By “significant” agonist activity is intended an agonist activity of at least 30%, 35%, 40%, 45%, 50%, 60%, 70%, 75%, 80%, 85%, 90%, 95%, or 100% greater than the agonist activity induced by a neutral substance or negative control as measured in an assay of a CD40-expressing cell's response. Preferably, “significant” agonist activity is an agonist activity that is at least 2-fold greater or at least 3-fold greater than the agonist activity induced by a neutral substance or negative control as measured in an assay of a B cell response. Thus, for example, where the B cell response of interest is B cell proliferation, “significant” agonist activity would be induction of a level of B cell proliferation that is at least 2-fold greater or at least 3-fold greater than the level of B cell proliferation induced by a neutral substance or negative control. In one embodiment, a non-specific immunoglobulin, for example IgG1, that does not bind to CD40 serves as the negative control. A substance “free of significant agonist activity” would exhibit an agonist activity of not more than about 25% greater than the agonist activity induced by a neutral substance or negative control, preferably not more than about 20% greater, 15% greater, 10% greater, 5% greater, 1% greater, 0.5% greater, or even not more than about 0.1% greater than the agonist activity induced by a neutral substance or negative control. For purposes of the present invention, the agonist activity of the anti-CD40 antibodies of the invention is measured in an assay of a B cell response. Such assays are well known in the art and include, but are not limited to, assays for monitoring immunoglobulin secretion by B cells, B cell proliferation assays, Banchereau-Like-B cell proliferation assays, co-stimulation of B cell proliferation assays. See, for example, such assays disclosed in Brodeur et al. (2003) Immunity 18:837, WO 00/75348, and U.S. Pat. No. 6,087,329, herein incorporated by reference. In one embodiment of the invention, the antagonist anti-CD40 antibody is free of significant agonist activity in one B cell response. In another embodiment of the invention, the antagonist anti-CD40 antibody is free of significant agonist activity in assays of more than one B cell response (e.g., proliferation and differentiation, or proliferation, differentiation, and antibody production).

In some embodiments, the antagonist anti-CD40 antibodies of the invention are fully human anti-CD40 monoclonal antibodies of the IgG1 isotype produced from a hybridoma cell line. These cell lines are created using splenocytes from immunized mice, including mice obtained using XenoMouse® technology (Abgenix; Fremont, Calif.), such as described in U.S. Pat. No. 6,075,181 and PCT International Publication No. WO 94/02602. The spleen cells are fused with the mouse myeloma SP2/0 cells (Sierra BioSource). The resulting hybridomas are sub-cloned several times to create the monoclonal cell lines. Other antibodies of the invention may be prepared similarly using mice transgenic for human immunoglobulin loci or by other methods known in the art and/or described herein. The human anti-CD40 monoclonal antibodies of the invention specifically bind CD40, for example, human CD40, though they may also specifically bind to a non-human sequence that has an epitope that the human anti-CD40 antibody recognizes.

In alternative embodiments, murine antibodies to CD40 can be humanized, for example, using methods described in U.S. Pat. No. 5,766,886 or U.S. Pat. No. 6,180,370. In addition, phage display libraries of human antibodies can be screened against C4BP to identify anti-CD40 antibodies having the binding characteristics described herein.

In addition to antagonist activity, some anti-CD40 antibodies of this invention have another mechanism of action, for example, antibodies having antibody-dependent cell-mediated cytotoxicity (ADCC). Alternatively, the variable regions of the anti-CD40 antibodies can be expressed on another antibody isotype that has ADCC activity. It is also possible to conjugate native forms, recombinant forms, or antigen-binding fragments of the anti-CD40 antibodies to a cytotoxic toxin. Such antagonist anti-CD40 antibodies are useful in targeting, for example, CD40-expressing neoplastic cells, for example, malignant B cells or CD40-expressing neoplastic cells of a solid tumor.

In some embodiments, the antagonist anti-CD40 antibodies of the invention, or antigen-binding fragments thereof, bind soluble CD40 in ELISA-type assays; in other embodiments, the antibodies or antigen binding fragments thereof inhibit binding of C4BP to cell-surface CD40, and thereby displace the pre-bound C4BP, as determined by flow cytometric assays. Suitable antagonist anti-CD40 antibodies or antigen-binding fragments thereof for use in the methods of the present invention exhibit a strong single-site binding affinity for the CD40 cell-surface antigen. In some embodiments, the antibodies of the invention exhibit a dissociation equilibrium constant (K_{D}) for CD40 of at least 10⁻⁵ M, at least 3×10⁻⁵ M, preferably at least 10⁻⁶ M to 10⁻⁷ M, more preferably at least 10⁻⁸ M to about 10⁻¹² M, measured using a standard assay such as Biacore™. Biacore analysis is known in the art and details are provided in the BIAapplications Handbook. Methods described in WO 01/27160 can be used to modulate the binding affinity.

Production of Antagonist Anti-CD40 Antibodies

The antagonist anti-CD40 antibodies of the invention include antibodies that specifically recognize the CD40 cell surface antigen, including polyclonal antibodies, monoclonal antibodies, single-chain antibodies, and fragments thereof such as Fab, F(ab′)₂, Fᵥ, and other fragments that retain the antigen binding function of the parent anti-CD40 antibody. Of particular interest to the methods of the present invention are those anti-CD40 antibodies that block C4BP-mediated CD40 cell signaling and are free of significant agonist activity when bound to CD40. These antibodies can be produced using any antibody production method known to those of skill in the art, and include antagonist anti-CD40 antibodies, and antigen-binding fragments thereof, that block C4BP-mediated CD40 signaling and which are recombinantly produced.

Polyclonal sera may be prepared by conventional methods. In general, a solution containing the CD40 antigen is first used to immunize a suitable animal, preferably a mouse, rat, rabbit, or goat. Rabbits or goats are preferred for the preparation of polyclonal sera due to the volume of serum obtainable, and the availability of labeled anti-rabbit and anti-goat antibodies.

Polyclonal sera can be prepared in a transgenic animal, preferably a mouse bearing human immunoglobulin loci. In a preferred embodiment, Sf9 cells expressing CD40 are used as the immunogen. Immunization can also be performed by mixing or emulsifying the antigen-containing solution in saline, preferably in an adjuvant such as Freund's complete adjuvant, and injecting the mixture or emulsion parenterally (generally subcutaneously or intramuscularly). A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization.

Polyclonal antisera are obtained by bleeding the immunized animal into a glass or plastic container, incubating the blood at 25° C. for one hour, followed by incubating at 4° C. for 2-18 hours. The serum is recovered by centrifugation (e.g., 1,000×g for 10 minutes). About 20-50 ml per bleed may be obtained from rabbits.

Production of the Sf9 (Spodoptera frugiperda) cells is disclosed in U.S. Pat. No. 6,004,552, incorporated herein by reference. Briefly, sequences encoding human CD40 are recombined into a baculovirus using transfer vectors. The plasmids are co-transfected with wild-type baculovirus DNA into Sf9 cells. Recombinant baculovirus-infected Sf9 cells are identified and clonally purified.

Preferably the antibody is monoclonal in nature. By “monoclonal antibody” is intended an antibody obtained from a population of substantially homogeneous antibodies, i.e., the individual antibodies comprising the population are identical except for possible naturally occurring mutations that may be present in minor amounts. The term is not limited regarding the species or source of the antibody. The term encompasses whole immunoglobulins as well as fragments such as Fab, F(ab′)2, Fv, and others that retain the antigen-binding function of the antibody. Monoclonal antibodies are highly specific, being directed against a single antigenic site, i.e., the CD40 cell surface antigen in the present invention. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in, for example, Clackson et al. (1991) Nature 352:624-628; Marks et al. (1991) J. Mol. Biol. 222:581-597; and U.S. Pat. No. 5,514,548.

By “epitope” is intended the part of an antigenic molecule to which an antibody is produced and to which the antibody will bind. Epitopes can comprise linear amino acid residues (i.e., residues within the epitope are arranged sequentially one after another in a linear fashion), nonlinear amino acid residues (referred to herein as “nonlinear epitopes”; these epitopes are not arranged sequentially), or both linear and nonlinear amino acid residues.

Monoclonal antibodies can be prepared using the method of Kohler et al. (1975) Nature 256:495-496, or a modification thereof. Typically, a mouse is immunized with a solution containing an antigen. Immunization can be performed by mixing or emulsifying the antigen-containing solution in saline, preferably in an adjuvant such as Freund's complete adjuvant, and injecting the mixture or emulsion parenterally. Any method of immunization known in the art may be used to obtain the monoclonal antibodies of the invention. After immunization of the animal, the spleen (and optionally, several large lymph nodes) are removed and dissociated into single cells. The spleen cells may be screened by applying a cell suspension to a plate or well coated with the antigen of interest. The B cells expressing membrane bound immunoglobulin specific for the antigen bind to the plate and are not rinsed away. Resulting B cells, or all dissociated spleen cells, are then induced to fuse with myeloma cells to form hybridomas, and are cultured in a selective medium. The resulting cells are plated by serial dilution and are assayed for the production of antibodies that specifically bind the antigen of interest (and that do not bind to unrelated antigens). The selected monoclonal antibody (mAb)-secreting hybridomas are then cultured either in vitro (e.g., in tissue culture bottles or hollow fiber reactors), or in vivo (as ascites in mice).

Where the antagonist anti-CD40 antibodies of the invention are to be prepared using recombinant DNA methods (i.e., recombinantly produced antagonist anti-CD40 antibodies), the DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells described herein serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al. (1993) Curr. Opinion in Immunol. 5:256 and Phickthun (1992) Immunol. Revs. 130:151.

Alternatively, antibody can be produced in a cell line such as a CHO cell line, as disclosed in U.S. Pat. Nos. 5,545,403; 5,545,405; and 5,998,144; incorporated herein by reference. Briefly the cell line is transfected with vectors capable of expressing a light chain and a heavy chain, respectively. By transfecting the two proteins on separate vectors, chimeric antibodies can be produced. Another advantage is the correct glycosylation of the antibody. In another embodiment, the antagonist anti-CD40 antibody or antigen-binding fragment thereof can be produced in CHO cells using the GS gene expression system (Lonza Biologics, Portsmouth, N.H.), which uses glutamine synthetase as a marker. See, also U.S. Pat. Nos. 5,122,464; 5,591,639; 5,658,759; 5,770,359; 5,827,739; 5,879,936; 5,891,693; and 5,981,216; the contents of which are herein incorporated by reference in their entirety.

Monoclonal antibodies to CD40 are known in the art. See, for example, the sections dedicated to B cell antigen in McMichael, ed. (1987; 1989) Leukocyte Typing III and IV (Oxford University Press, New York); U.S. Pat. Nos. 5,674,492; 5,874,082; 5,677,165; 6,056,959; International Publication Nos. WO 00/63395, WO 02/28905, and WO 02/28904; U.S. Patent Application Publication Nos. US 2002/0142358 A1 and 2003/0059427; the antagonist anti-CD40 antibodies disclosed in provisional applications entitled “Antagonist Anti-CD40 Monoclonal Antibodies and Methods for Their Use,” filed Nov. 4, 2003, Nov. 26, 2003, and Apr. 27, 2004, and assigned U.S. Patent Application Nos. 60/517,337 (Attorney Docket No. PP20107.001 (035784/258442)), 60/525,579 (Attorney Docket No. PP20107.002 (035784/271525)), and 60/565,710 (Attorney Docket No. PP20107.003 (035784/277214)), respectively, and International Patent Application No. PCT/US2004/037152 (Attorney Docket No. PP20107.004 (035784/282916)), also entitled “Antagonist Anti-CD40 Monoclonal Antibodies and Methods for Their Use,” filed Nov. 4, 2004; Gordon et al. (1988) J. Immunol. 140:1425; Valle et al. (1989) Eur. J. Immunol. 19:1463; Clark et al. (1986) PNAS 83:4494; Paulie et al. (1989) J. Immunol. 142:590; Gordon et al. (1987) Eur. J. Immunol. 17:1535; Jabara et al. (1990) J. Exp. Med. 172:1861; Zhang et al. (1991) J. Immunol. 146:1836; Gascan et al. (1991) J. Immunol. 147:8; Banchereau et al. (1991) Clin. Immunol. Spectrum 3:8; and Banchereau et al. (1991) Science 251:70; all of which are herein incorporated by reference. Of particular interest to the present invention are the antagonist anti-CD40 antibodies disclosed herein that bind at or near the C4BP binding site on CD40, thereby blocking CD40-mediated CD40 signaling, which can be the result of the antagonist anti-CD40 antibodies' ability to competitively or sterically inhibit the binding of C4BP to its binding site on C4BP or their ability to prevent CD40 signal transduction with the binding of C4BP to its binding site on CD40.

The term “CD40-antigen epitope” as used herein refers to a molecule that is capable of immunoreactivity with the anti-CD40 monoclonal antibodies of this invention, excluding the CD40 antigen itself. CD40-antigen epitopes may comprise proteins, protein fragments, peptides, carbohydrates, lipids, and other molecules, but for the purposes of the present invention are most commonly proteins, short oligopeptides, oligopeptide mimics (i e, organic compounds which mimic the antibody binding properties of the CD40 antigen), or combinations thereof. Suitable oligopeptide mimics are described, inter alia, in PCT application US 91/04282.

Additionally, the antagonist anti-CD40 antibodies of the invention include chimeric anti-CD40 antibodies and humanized anti-CD40 antibodies; such chimeric anti-CD40 antibodies and humanized anti-CD40 antibodies of the invention bind at or near the C4BP binding site on CD40, thereby blocking C4BP-mediated CD40 signaling. By “chimeric” antibodies is intended antibodies that are most preferably derived using recombinant deoxyribonucleic acid techniques and which comprise both human (including immunologically “related” species, e.g., chimpanzee) and non-human components. Thus, the constant region of the chimeric antibody is most preferably substantially identical to the constant region of a natural human antibody; the variable region of the chimeric antibody is most preferably derived from a non-human source and has the desired antigenic specificity to the CD40 cell-surface antigen. The non-human source can be any vertebrate source that can be used to generate antibodies to a human CD40 cell-surface antigen or material comprising a human CD40 cell-surface antigen. Such non-human sources include, but are not limited to, rodents (e.g., rabbit, rat, mouse, etc.; see, for example, U.S. Pat. No. 4,816,567, herein incorporated by reference) and non-human primates (e.g., Old World Monkey, Ape, etc.; see, for example, U.S. Pat. Nos. 5,750,105 and 5,756,096; herein incorporated by reference). Rituxan® is an example of a chimeric antibody with a murine variable region and a human constant region. As used herein, the phrase “immunologically active” when used in reference to chimeric anti-CD40 antibodies means a chimeric antibody that binds CD40, particularly human CD40.

By “humanized” is intended forms of anti-CD40 antibodies that contain minimal sequence derived from non-human immunoglobulin sequences. For the most part, humanized antibodies are human immunoglobulins (recipient antibody) in which residues from a hypervariable region (also known as complementarity determining region or CDR) of the recipient are replaced by residues from a hypervariable region of a non-human species (donor antibody) such as mouse, rat, rabbit, or nonhuman primate having the desired specificity, affinity, and capacity. The phrase “complementarity determining region” refers to amino acid sequences that together define the binding affinity and specificity of the natural Fv region of a native immunoglobulin binding site. See, e.g., Chothia et al (1987) J. Mol. Biol. 196:901-917; Kabat et al (1991) U.S. Dept. of Health and Human Services, NIH Publication No. 91-3242). The phrase “constant region” refers to the portion of the antibody molecule that confers effector functions. In previous work directed towards producing non-immunogenic antibodies for use in therapy of human disease, mouse constant regions were substituted by human constant regions. The constant regions of the subject humanized antibodies were derived from human immunoglobulins. However, these humanized antibodies still elicited an unwanted and potentially dangerous immune response in humans and there was a loss of affinity. Humanized anti-CD40 antibodies of the present invention also specifically bind to CD40, thereby blocking or inhibiting C4BP-mediated CD40 signaling.

Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting rodent or mutant rodent CDRs or CDR sequences for the corresponding sequences of a human antibody. See also U.S. Pat. Nos. 5,225,539; 5,585,089; 5,693,761; 5,693,762; 5,859,205; herein incorporated by reference. In some instances, residues within the framework regions of one or more variable regions of the human immunoglobulin are replaced by corresponding non-human residues (see, for example, U.S. Pat. Nos. 5,585,089; 5,693,761; 5,693,762; and 6,180,370). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al. (1986) Nature 331:522-525; Riechmann et al. (1988) Nature 332:323-329; and Presta (1992) Curr. Op. Struct. Biol. 2:593-596; herein incorporated by reference. Accordingly, such “humanized” antibodies may include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and International Publication No. WO 01/27160, where humanized antibodies and techniques for producing humanized antibodies having improved affinity for a predetermined antigen are disclosed.

The anti-CD40 antibodies of the present invention also include xenogeneic or modified anti-CD40 antibodies produced in a non-human mammalian host, more particularly a transgenic mouse, characterized by inactivated endogenous immunoglobulin (Ig) loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. Nos. 5,877,397 and 5,939,598, herein incorporated by reference. Accordingly, such antibodies are fully human anti-CD40 antibodies.

Preferably, fully human antibodies to CD40 are obtained by immunizing transgenic mice. One such mouse is obtained using Xenomouse® technology (Abgenix; Fremont, Calif.), and is disclosed in U.S. Pat. Nos. 6,075,181, 6,091,001, and 6,114,598, all of which are incorporated herein by reference. Thus, for example, in one embodiment, the human antagonist anti-CD40 antibodies disclosed herein can be produced by immunizing mice transgenic for the human IgG₁ heavy chain locus and the human κ light chain locus with Sf9 cells expressing human CD40. Mice can also be transgenic for other isotypes. Fully human antagonist anti-CD40 antibodies of the present invention are also characterized by binding at or near the C4BP binding site on CD40, thereby blocking C4BP-mediated CD40 signaling.

Fragments of the antagonist anti-CD40 antibodies of the present invention are suitable for use in the methods of the invention so long as they retain the desired affinity of the corresponding full-length antagonist anti-CD40 antibody and are characterized by properties similar to the corresponding full-length antagonist anti-CD40 antibody. That is, the fragments will specifically bind a CD40 antigen expressed on the surface of a cell, for example, human CD40 antigen on the surface of a human cell, and are free of significant agonist activity but exhibit antagonist activity when bound to the CD40 antigen. Accordingly, binding of such fragments to CD40 on CD40-expressing cells blocks C4BP-mediated CD40 signaling, thereby inhibiting one or more CD40-directed activities. Such fragments are referred to herein as “antigen-binding” fragments, and are suitable for use in any of the methods of the present invention.

Suitable antigen-binding fragments of an antibody comprise a portion of a full-length antibody, generally the antigen-binding or variable region thereof. Examples of antibody fragments include, but are not limited to, Fab, F(ab′)₂, and Fv fragments and single-chain antibody molecules. By “Fab” is intended a monovalent antigen-binding fragment of an immunoglobulin that is composed of the light chain and part of the heavy chain. By F(ab′)₂ is intended a bivalent antigen-binding fragment of an immunoglobulin that contains both light chains and part of both heavy chains. By “single-chain Fv” or “sFv” antibody fragments is intended fragments comprising the V_{H} and V_{L} domains of an antibody, wherein these domains are present in a single polypeptide chain. See, for example, U.S. Pat. Nos. 4,946,778, 5,260,203, 5,455,030, and 5,856,456, herein incorporated by reference. Generally, the Fv polypeptide further comprises a polypeptide linker between the V_{H} and V_{L} domains that enables the sFv to form the desired structure for antigen binding. For a review of sFv see Pluckthun (1994) in The Pharmacology of Monoclonal Antibodies, Vol. 113, ed. Rosenburg and Moore (Springer-Verlag, New York), pp. 269-315. Antigen-binding fragments of the antagonist anti-CD40 antibodies disclosed herein can also be conjugated to a cytotoxin to effect killing of target cells, for example, target cancer cells, as described herein below.

Antibodies or antibody fragments can be isolated from antibody phage libraries generated using the techniques described in, for example, McCafferty et al. (1990) Nature 348:552-554 (1990) and U.S. Pat. No. 5,514,548. Clackson et al. (1991) Nature 352:624-628 and Marks et al. (1991) J. Mol. Biol. 222:581-597 describe the isolation of murine and human antibodies, respectively, using phage libraries. Subsequent publications describe the production of high affinity (nM range) human antibodies by chain shuffling (Marks et al. (1992) Bio/Technology 10:779-783), as well as combinatorial infection and in vivo recombination as a strategy for constructing very large phage libraries (Waterhouse et al. (1993) Nucleic. Acids Res. 21:2265-2266). Thus, these techniques are viable alternatives to traditional monoclonal antibody hybridoma techniques for isolation of monoclonal antibodies.

Various techniques have been developed for the production of antibody fragments. Traditionally, these fragments were derived via proteolytic digestion of intact antibodies (see, e.g., Morimoto et al. (1992) Journal of Biochemical and Biophysical Methods 24:107-117 (1992) and Brennan et al. (1985) Science 229:81). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al. (1992) Bio/Technology 10:163-167). According to another approach, F(ab′)₂ fragments can be isolated directly from recombinant host cell culture. Other techniques for the production of antibody fragments will be apparent to the skilled practitioner.

Antagonist anti-CD40 antibodies useful in the methods of the present invention include the anti-CD40 monoclonal antibodies described herein above as well as antibodies differing from these antibodies but retaining the CDRs; and antibodies with one or more amino acid addition(s), deletion(s), or substitution(s), wherein the antagonist activity is measured by the ability of the antibody to block C4BP-mediated CD40 signaling, thereby inhibiting one or more CD40-directed activities. The invention also encompasses de-immunized antagonist anti-CD40 antibodies, which can be produced as described in, for example, International Publication Nos. WO 98/52976 and WO 0034317; herein incorporated by reference. In this manner, residues within the antagonist anti-CD40 antibodies of the invention are modified so as to render the antibodies non- or less immunogenic to humans while retaining their antagonist activity toward human CD40-expressing cells, particularly blocking C4BP-mediated CD40 signaling, resulting in inhibition of one or more CD40-directed activities, wherein such activities are measured by assays noted elsewhere herein. Also included within the scope of the claims are fusion proteins comprising an antagonist anti-CD40 antibody of the invention, or a fragment thereof, which fusion proteins can be synthesized or expressed from corresponding polynucleotide vectors, as is known in the art. Such fusion proteins are described with reference to conjugation of antibodies as noted below.

The antibodies of the present invention can have sequence variations produced using methods described in, for example, Patent Publication Nos. EP 0 983 303 A1, WO 00/34317, and WO 98/52976, incorporated herein by reference. For example, it has been shown that sequences within the CDR can cause an antibody to bind to MHC Class II and trigger an unwanted helper T-cell response. A conservative substitution can allow the antibody to retain binding activity yet lose its ability to trigger an unwanted T-cell response. Any such conservative or non-conservative substitutions can be made using art-recognized methods, such as those noted elsewhere herein, and the resulting antibodies will fall within the scope of the invention. The variant antibodies can be routinely tested for antagonist activity, affinity, and specificity using methods described herein.

An antibody produced by any of the methods described above, or any other method not disclosed herein, will fall within the scope of the invention if the binding of the antibody to CD40 antigen blocks C4BP-mediated CD40 signaling. The antibody can block C4BP-mediated CD40 signaling by any means including, but not limited to: preventing binding of C4BP by sterically inhibiting the binding of C4BP to its binding site on CD40, competitively inhibiting binding of C4BP by competing for at least a portion of the C4BP binding site on CD40, and preventing CD40 signal transduction when C4BP ligates cell surface CD40. The antagonistic anti-CD40 antibodies of this invention may thus inhibit one or more of the CD40-directed activities that is induced by the binding of C4BP to its binding site on CD40, including, but not limited to, the CD40-directed activities disclosed herein, for example, immunoglobulin secretion by normal human peripheral B cells stimulated by T cells; survival and/or proliferation of normal human peripheral B cells stimulated by Jurkat T cells; survival and/or proliferation of normal human peripheral B cells stimulated by C4BP-expressing cells or soluble C4BP; “survival” anti-apoptotic intracellular signals in any cell stimulated by soluble C4BP or solid-phase C4BP; CD40 signal transduction in any cell upon ligation with soluble C4BP or solid-phase C4BP; and proliferation of human malignant B cells as noted below. Assays for determining the ability of an antagonist anti-CD40 antibody to inhibit these CD40-directed activities can be performed as described in the Examples herein. See, also, the assays described in Schultze et al. (1998) Proc. Natl. Acad. Sci. USA 92:8200; Denton et al. (1998) Pediatr. Transplant. 2:6-15; Evans et al. (2000) J. Immunol. 164:688; Noelle (1998) Agents Actions Suppl. 49:17-22; Lederman et al. (1996) Curr. Opin. Hematol. 3:77; Coligan et al. (1991) Current Protocols in Immunology 13:12; Kwekkeboom et al. (1993) Immunology 79:439; and U.S. Pat. Nos. 5,674,492 and 5,847,082; herein incorporated by reference.

A representative assay to detect antagonistic anti-CD40 antibodies that specifically bind to the CD40 antigen and block C4BP-mediated CD40 signaling in the manner identified herein is a “competitive binding assay.” Competitive binding assays are serological assays in which unknowns are detected and quantitated by their ability to inhibit the binding of a labeled known ligand, such as C4BP, to its specific receptor such as CD40. This is also referred to as a competitive inhibition assay. In a representative competitive binding assay, labeled CD40 polypeptide is precipitated by candidate antibodies in a sample, for example, in combination with monoclonal antibodies raised against one or more epitopes of the antibodies of the invention. Anti-CD40 antibodies that specifically bind an epitope of interest can be identified by screening a series of antibodies prepared against a CD40 protein or fragment of the protein comprising the particular epitope of the CD40 protein of interest. For example, for human CD40, epitopes of interest include epitopes comprising linear and/or nonlinear amino acid residues of the short isoform of human CD40 (see GenBank Accession No. NP_{—}690593, encoded by GenBank Accession No. NM_{—}152854), or of the long isoform of human CD40 (see GenBank Accession Nos. CAA43045 and NP_{—}001241, encoded by GenBank Accession Nos. X60592 and NM_{—}001250). Alternatively, competitive binding assays with previously identified suitable antagonist anti-CD40 antibodies could be used to select monoclonal antibodies comparable to the previously identified antibodies.

Antibodies employed in such immunoassays may be labeled or unlabeled. Unlabeled antibodies may be employed in agglutination or ELISA; labeled antibodies may be employed in a wide variety of assays, employing a wide variety of labels. Detection of the formation of an antibody-antigen complex between an anti-CD40 antibody and an epitope of interest can be facilitated by attaching a detectable substance to the antibody. Suitable detection means include the use of labels such as radionuclides, enzymes, coenzymes, fluorescers, chemiluminescers, chromogens, enzyme substrates or co-factors, enzyme inhibitors, prosthetic group complexes, free radicals, particles, dyes, and the like. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin; and examples of suitable radioactive material include ¹²⁵I, ¹³¹I, ³⁵S, or ³H. Such labeled reagents may be used in a variety of well-known assays, such as radioimmunoassays, enzyme immunoassays, e.g., ELISA, fluorescent immunoassays, and the like. See for example, U.S. Pat. Nos. 3,766,162; 3,791,932; 3,817,837; and 4,233,402.

Any of the previously described antagonist anti-CD40 antibodies or antigen-binding fragments thereof may be conjugated prior to use in the methods of the present invention. Methods for producing conjugated antibodies are known in the art. Thus, the anti-CD40 antibody may be labeled using an indirect labeling or indirect labeling approach. By “indirect labeling” or “indirect labeling approach” is intended that a chelating agent is covalently attached to an antibody and at least one radionuclide is inserted into the chelating agent. See, for example, the chelating agents and radionuclides described in Srivastava and Mease (1991) Nucl. Med. Bio. 18:589-603, herein incorporated by reference. Suitable labels include fluorophores, chromophores, radioactive atoms (particularly ³²P and ¹²⁵I, electron-dense reagents, enzymes, and ligands having specific binding partners. Enzymes are typically detected by their activity. For example, horseradish peroxidase is usually detected by its ability to convert 3,3′,5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefore. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, ¹²⁵I may serve as a radioactive label or as an electron-dense reagent. Horseradish peroxidase (HRP) may serve as enzyme or as antigen for a monoclonal antibody. Further, one may combine various labels for desired effect. For example, monoclonal antibodies and avidin also require labels in the practice of this invention: thus, one might label a monoclonal antibody with biotin, and detect its presence with avidin labeled with ¹²⁵I, or with an anti-biotin monoclonal antibody labeled with HRP. Other permutations and possibilities will be readily apparent to those of ordinary skill in the art, and are considered as equivalents within the scope of the instant invention.

Alternatively, the anti-(CD40 antibody may be labeled using “direct labeling” or a “direct labeling approach,” where a radionuclide is covalently attached directly to an antibody (typically via an amino acid residue). Preferred radionuclides are provided in Srivagtava and Mease (1991) supra. The indirect labeling approach is particularly preferred. See also, for example, International Publication Nos. WO 00/52031 and WO 00/52473, where a linker is used to attach a radioactive label to antibodies; and the labeled forms of anti-CD40 antibodies described in U.S. Pat. No. 6,015,542; herein incorporated by reference.

Further, an antibody (or fragment thereof) may be conjugated to a therapeutic moiety such as a cytotoxin, a therapeutic agent, or a radioactive metal ion or radioisotope. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Radioisotopes include, but are not limited to, I-131, 1-123, I-125, Y-90, Re-188, Re-186, At-211, Cu-67, Bi-212, Bi-213, Pd-109, Tc-99, In-111, and the like. The conjugated antibodies of the invention can be used for modifying a given biological response; the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, interferon-alpha, interferon-beta, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known. See, for example, Arnon et al. (1985) “Monoclonal Antibodies for Immunotargeting of Drugs in Cancer Therapy,” in Monoclonal Antibodies and Cancer Therapy, ed. Reisfeld et al. (Alan R. Liss, Inc.), pp. 243-256; Hellstrom et al. (1987) “Antibodies for Drug Delivery,” in Controlled Drug Delivery, ed. Robinson et al. (2d ed; Marcel Dekker, Inc.), pp. 623-653; Thorpe (1985) “Antibody Carriers of Cytotoxic Agents in Cancer Therapy: A Review,” in Monoclonal Antibodies '84: Biological and Clinical Applications, ed. Pinchera et al. (Editrice Kurtis, Milano, Italy, 1985), pp. 475-506; “Analysis, Results, and Future Prospective of the Therapeutic Use of Radiolabeled Antibody in Cancer Therapy,” in Monoclonal Antibodies for Cancer Detection and Therapy, ed. Baldwin et al. (Academic Press, New York, 1985), pp. 303-316; and Thorpe et al. (1982) Immunol. Rev. 62:119-158.

Alternatively, an antibody can be conjugated to a second antibody to form an antibody heteroconjugate as described in U.S. Pat. No. 4,676,980. In addition, linkers may be used between the labels and the antibodies of the invention (see U.S. Pat. No. 4,831,175). Antibodies, or antigen-binding fragments thereof, may be directly labeled with radioactive iodine, indium, yttrium, or other radioactive particle known in the art (U.S. Pat. No. 5,595,721). Treatment may consist of a combination of treatment with conjugated and nonconjugated antibodies administered simultaneously or subsequently (International Publication Nos. WO 00/52031 and WO 00/52473).

Variants of Antagonist Anti-CD40 Antibodies

Suitable biologically active variants of the antagonist anti-CD40 antibodies can be used in the methods of the present invention. Such variants will retain the desired binding properties of the parent antagonist anti-CD40 antibody. Methods for making antibody variants are generally available in the art.

For example, amino acid sequence variants of an antagonist anti-CD40 antibody can be prepared by mutations in the cloned DNA sequence encoding the antibody of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488; Kunkel et al. (1987) Methods Enzymol. 154:367; Sambrook et al. (1989) Molecular Cloning: A Laboratory Manual (Cold Spring Harbor, N.Y.); U.S. Pat. No. 4,873,192; and the references cited therein; herein incorporated by reference. Guidance as to appropriate amino acid substitutions that do not affect biological activity of the polypeptide of interest may be found in the model of Dayhoff et al. (1978) in Atlas of Protein Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr.

In constructing variants of the antagonist anti-CD40 antibody polypeptide of interest, modifications are made such that variants continue to possess the desired activity, i.e., similar binding affinity and are capable of specifically binding to a human CD40 antigen expressed on the surface of a human cell thereby blocking C4BP-mediated CD40 signaling, and being free of significant agonist activity but exhibiting antagonist activity when bound to a CD40 antigen on a human CD40-expressing cell. Obviously, any mutations made in the DNA encoding the variant polypeptide must not place the sequence out of reading frame and preferably will not create complementary regions that could produce secondary mRNA structure. See EP Patent Application Publication No. 75,444.

In addition, the constant region of an antagonist anti-CD40 antibody can be mutated to alter effector function in a number of ways. For example, see U.S. Pat. No. 6,737,056B1 and U.S. Patent Application Publication No. 2004/0132101A1, which disclose Fc mutations that optimize antibody binding to Fc receptors.

Preferably, variants of a reference antagonist anti-CD40 antibody have amino acid sequences that have at least 70% or 75% sequence identity, preferably at least 80% or 85% sequence identity, more preferably at least 90%, 91%, 92%, 93%, 94% or 95% sequence identity to the amino acid sequence for the reference antagonist anti-CD40 antibody molecule, or to a shorter portion of the reference antibody molecule. More preferably, the molecules share at least 96%, 97%, 98% or 99% sequence identity. For purposes of the present invention, percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv. Appl. Math. 2:482-489. A variant may, for example, differ from the reference antagonist anti-CD40 antibody by as few as 1 to 15 amino acid residues, as few as 1 to 10 amino acid residues, such as 6-10, as few as 5, as few as 4, 3, 2, or even 1 amino acid residue.

With respect to optimal alignment of two amino acid sequences, the contiguous segment of the variant amino acid sequence may have additional amino acid residues or deleted amino acid residues with respect to the reference amino acid sequence. The contiguous segment used for comparison to the reference amino acid sequence will include at least 20 contiguous amino acid residues, and may be 30, 40, 50, or more amino acid residues. Corrections for sequence identity associated with conservative residue substitutions or gaps can be made (see Smith-Waterman homology search algorithm).

The precise chemical structure of an anti-CD40 antibody capable of specifically binding CD40 and retaining antagonist activity, particularly when bound to CD40 antigen, depends on a number of factors. As ionizable amino and carboxyl groups are present in the molecule, a particular polypeptide may be obtained as an acidic or basic salt, or in neutral form. All such preparations that retain their biological activity when placed in suitable environmental conditions are included in the definition of antagonist anti-CD40 antibodies as used herein. Further, the primary amino acid sequence of the polypeptide may be augmented by derivatization using sugar moieties (glycosylation) or by other supplementary molecules such as lipids, phosphate, acetyl groups and the like. It may also be augmented by conjugation with saccharides. Certain aspects of such augmentation are accomplished through post-translational processing systems of the producing host; other such modifications may be introduced in vitro. In any event, such modifications are included in the definition of an anti-CD40 antibody used herein so long as the antagonist properties of the anti-CD40 antibody are not destroyed. It is expected that such modifications may quantitatively or qualitatively affect the activity, either by enhancing or diminishing the activity of the antibody, in the various assays. Further, individual amino acid residues in the chain may be modified by oxidation, reduction, or other derivatization, and the antibody may be cleaved to obtain fragments that retain activity. Such alterations that do not destroy antagonist activity do not remove the antibody polypeptide sequence from the definition of anti-CD40 antibodies of interest as used herein.

The art provides substantial guidance regarding the preparation and use of variants of antibodies. In preparing the anti-CD40 antibody variants, one of skill in the art can readily determine which modifications to the native nucleotide or amino acid sequence will result in a variant that is suitable for use as a therapeutically active component of a pharmaceutical composition used in the methods of the present invention.

Methods of Therapy Using the Antagonist Anti-CD40 Antibodies of the Invention

As the antagonist anti-CD40 antibodies provide a means for blocking C4BP-mediated CD40 signaling, they can be used to inhibit one or more CD40-directed activities as noted herein above. Thus the present invention provides a method for inhibiting a CD40-directed activity in a CD40-expressing cell, where the method comprises contacting the cell with an amount of an antagonist anti-CD40 antibody of the invention effective to block C4BP-mediated CD40 signaling. As previously noted, blocking of this signaling process can be the result of competitive inhibition or steric inhibition of the binding of C4BP to its binding site on CD40, or prevention of CD40 signal transduction with the binding of C4BP to its binding site on CD40, so long as binding of the anti-CD40 antibody to CD40 prevents C4BP-mediated CD40 signaling.

The antagonist anti-CD40 antibodies disclosed herein can be used to treat patients having a disease mediated by C4BP stimulation of CD40 signaling on CD40-expressing cells. By “CD40-expressing cell” is intended any cell type that expresses the CD40 cell surface antigen, particularly B cells and other APCs, including dendritic cells, and can be normal or malignant CD40-expressing cells. Methods for detecting CD40 expression in cells are well known in the art and include, but are not limited to, PCR techniques, immunohistochemistry, flow cytometry, Western blot, ELISA, and the like.

By “malignant B cell” is intended any neoplastic B cell, including but not limited to B cells derived from lymphomas including low-, intermediate-, and high-grade B cell lymphomas, immunoblastic lymphomas, non-Hodgkin's lymphomas, Hodgkin's disease, Epstein-Barr Virus (EBV) induced lymphomas, and AIDS-related lymphomas, as well as B cell acute lymphoblastic leukemias (ALLs), myelomas, chronic lymphocytic leukemias (CLLs), acute myeloblastic leukemias, and the like. In other embodiments, the CD40-expressing cell is a solid tumor cell. By “CD40-expressing solid tumor cell” is intended any malignant or pre-malignant cell of a solid tumor that expresses the CD40 cell-surface antigen. For purposes of the present invention, cancerous and precancerous or pre-malignant cells that express the CD40 antigen are referred to as “CD40-expressing neoplastic cells.” Further, where CD40 ligand (CD40L) and C4BP act synergistically via CD40 activation, the anti-CD40 antibodies of the invention can be used to block C4BP-mediated CD40 signaling, thereby having an effect on diseases that are mediated by CD40/CD40L engagement.

“Treatment” is herein defined as the application or administration of an antagonist anti-CD40 antibody or antigen-binding fragment thereof to a patient, or application or administration of an antagonist anti-CD40 antibody or fragment thereof to an isolated tissue or cell line from a patient, where the patient has a disease, a symptom of a disease, or a predisposition toward a disease, where the purpose is to cure, heal, alleviate, relieve, alter, remedy, ameliorate, improve, or affect the disease, the symptoms of the disease, or the predisposition toward the disease. By “treatment” is also intended the application or administration of a pharmaceutical composition comprising the antagonist anti-CD40 antibodies or fragments thereof to a patient, or application or administration of a pharmaceutical composition comprising the anti-CD40 antibodies or fragments thereof to an isolated tissue or cell line from a patient, who has a disease, a symptom of a disease, or a predisposition toward a disease, where the purpose is to cure, heal, alleviate, relieve, alter, remedy, ameliorate, improve, or affect the disease, the symptoms of the disease, or the predisposition toward the disease.

Therapy with at least one antagonist anti-CD40 antibody (or antigen-binding fragment thereof) of the present invention causes a physiological response that is beneficial with respect to treatment of diseases associated with C4BP stimulation of CD40 signaling on CD40-expressing cells in a human, referred to herein as CD40-associated diseases. Such CD40-associated diseases include, but are not limited to, hyperproliferative disorders, pre-malignant conditions, which may lead to cancers, cancers, including B cell-related cancers and solid tumors comprising CD40-expressing neoplastic cells, and autoimmune and/or inflammatory diseases. Thus, the antagonist anti-CD40 antibodies of the invention could be used to treat autoimmune and/or inflammatory diseases such as systemic lupus, psoriasis, multiple sclerosis, inflammatory bowel disease (Crohn's disease), rheumatoid arthritis, and rejection of organ and tissue transplants, by suppressing autoimmune response, to treat lymphomas by depriving malignant B lymphocytes of the activating signal provided by CD40, and to deliver toxins to CD40-bearing cells in a specific manner.

Thus, for example, the antagonist anti-CD40 antibodies of the invention find use in the treatment of non-Hodgkin's lymphomas related to abnormal, uncontrollable B cell proliferation or accumulation. For purposes of the present invention, such lymphomas will be referred to according to the Working Formulation classification scheme, that is those B cell lymphomas categorized as low grade, intermediate grade, and high grade (see “The Non-Hodgkin's Lymphoma Pathologic Classification Project,” (1982) Cancer 49:2112). Thus, low-grade B cell lymphomas include small lymphocytic, follicular small-cleaved cell, and follicular mixed small-cleaved and large cell lymphomas; intermediate-grade lymphomas include follicular large cell, diffuse small cleaved cell, diffuse mixed small and large cell, and diffuse large cell lymphomas; and high-grade lymphomas include large cell immunoblastic, lymphoblastic, and small non-cleaved cell lymphomas of the Burkitt's and non-Burkitt's type.

It is recognized that the antagonist anti-CD40 antibodies of the invention are useful in the therapeutic treatment of B cell lymphomas that are classified according to the Revised European and American Lymphoma Classification (REAL) system. Such B cell lymphomas include, but are not limited to, lymphomas classified as precursor B cell neoplasms, such as B lymphoblastic leukemia/lymphoma; peripheral B cell neoplasms, including B cell chronic lymphocytic leukemia/small lymphocytic lymphoma, lymphoplasmacytoid lymphoma/immunocytoma, mantle cell lymphoma (MCL), follicle center lymphoma (follicular) (including diffuse small cell, diffuse mixed small and large cell, and diffuse large cell lymphomas), marginal zone B cell lymphoma (including extranodal, nodal, and splenic types), hairy cell leukemia, plasmacytoma/myeloma, diffuse large cell B cell lymphoma of the subtype primary mediastinal (thymic), Burkitt's lymphoma, and Burkitt's like high grade B cell lymphoma; acute leukemias; acute lymphocytic leukemias (ALLs); myeloblastic leukemias; acute myelocytic leukemias; promyelocytic leukemia; myelomonocytic leukemia; monocytic leukemia; erythroleukemia; granulocytic leukemia (chronic myelocytic leukemia); chronic lymphocytic leukemia (CLL); polycythemia vera; multiple myeloma; Waldenstrom's macroglobulinemia; heavy chain disease; and unclassifiable low-grade or high-grade B cell lymphomas.

The antagonist anti-CD40 antibodies of the invention may be useful in preventing further tumor outgrowths arising during therapy, and can be useful in the treatment of subjects having low-grade B cell lymphomas, particularly those subjects having relapses following standard chemotherapy. Low-grade B cell lymphomas are more indolent than the intermediate- and high-grade B cell lymphomas and are characterized by a relapsing/remitting course. Thus, treatment of these lymphomas is improved using the antagonist anti-CD40 antibodies of the invention, as relapse episodes can be reduced in number and severity.

Solid tumors that comprise CD40-expressing neoplastic cells include, but are not limited to, ovarian, lung (for example, non-small cell lung cancer of the squamous cell carcinoma, adenocarcinoma, and large cell carcinoma types, and small cell lung cancer), breast, colon, kidney (including, for example, renal cell carcinomas), bladder (for example, urinary bladder carcinoma), liver (including, for example, hepatocellular carcinomas), gastric, cervical, prostate, nasopharyngeal, thyroid (for example, thyroid papillary carcinoma), and skin cancers such as melanoma, and sarcomas (including, for example, osteosarcomas and Ewing's sarcomas).

When administered to a subject having a cancer comprising CD40-expressing neoplastic cells, the antagonist anti-CD40 antibodies of the invention, and antigen-binding fragments thereof, can provide anti-tumor activity. By “anti-tumor activity” is intended a reduction in the rate of CD40-expressing neoplastic cell proliferation or accumulation, and hence a decline in growth rate of an existing tumor or in a tumor that arises during therapy, and/or destruction of existing neoplastic (tumor) cells or newly formed neoplastic cells, and hence a decrease in the overall size of a tumor during therapy.

Thus, the present invention provides methods for treating a cancer comprising CD40-expressing cells, such as the B cell lymphomas and solid tumors, wherein a therapeutically effective amount of an antagonist anti-CD40 antibody of the present invention, or antigen-binding fragment thereof, is administered to a subject having the cancer. Administration of these antibodies, or antigen-binding fragment thereof, promotes a positive therapeutic response. By “positive therapeutic response” with respect to cancer treatment is intended an improvement in the disease in association with the anti-tumor activity of these antibodies or fragments thereof, and/or an improvement in the symptoms associated with the disease. Thus, for example, a positive therapeutic response would refer to one or more of the following improvements in the disease: (1) a reduction in tumor size; (2) a reduction in the number of cancer (i.e., neoplastic) cells; (3) an increase in neoplastic cell death; (4) inhibition of neoplastic cell survival; (4) inhibition (i.e., slowing to some extent, preferably halting) of tumor growth; (5) inhibition (i.e., slowing to some extent, preferably halting) of cancer cell infiltration into peripheral organs; (6) inhibition (i.e., slowing to some extent, preferably halting) of tumor metastasis; (7) the prevention of further tumor outgrowths; (8) an increased patient survival rate; and (9) some extent of relief from one or more symptoms associated with the cancer. Such therapeutic responses may be further characterized as to degree of improvement. Thus, for example, an improvement in the disease may be characterized as a complete response. By “complete response” is intended an absence of clinically detectable disease with normalization of any previously abnormal radiographic studies, bone marrow, and cerebrospinal fluid (CSF). Such a response must persist for at least one month following treatment according to the methods of the invention. Alternatively, an improvement in the cancer may be categorized as being a partial response. By “partial response” is intended at least about a 50% decrease in all measurable tumor burden (i.e., the number of tumor cells present in the subject) in the absence of new lesions and persisting for at least one month. Such a response is applicable to measurable tumors only.

Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bioluminescent imaging, for example, luciferase imaging, bone scan imaging, and tumor biopsy sampling including bone marrow aspiration (BMA). In addition to these positive therapeutic responses, the subject undergoing therapy with the antagonist anti-CD40 antibody or antigen-binding fragment thereof may experience the beneficial effect of an improvement in the symptoms associated with the disease. Thus for B cell tumors, the subject may experience a decrease in the so-called B symptoms, i.e., night sweats, fever, weight loss, and/or urticaria.

The antagonist anti-CD40 antibodies described herein may also find use in the treatment of other CD40-associated diseases where blocking of C4BP-mediated CD40 signaling results in inhibition of one or more CD40-directed activities, and for which such inhibition results in an improvement in the disease, or at least reduces one or more undesirable symptoms of the disease. Thus, where C4BP-mediated CD40 signaling is associated with an undesirable immune response or process in vivo, such as occurs with diseases or disorders having an autoimmune and/or inflammatory component, an antagonist anti-CD40 antibody of the invention can be administered to an at-risk subject or subject in need of treatment for one or more of these CD40-associated diseases in order to block C4BP-mediated CD40 signaling, thereby inhibiting or preventing the symptoms associated with the respective CD40-associated disease.

Inflammatory diseases are characterized by inflammation and tissue destruction, or a combination thereof. “Inflammatory disease” includes any inflammatory immune-mediated process where the initiating event or target of the immune response involves non-self antigen(s), including, for example, alloantigens, xenoantigens, viral antigens, bacterial antigens, unknown antigens, or allergens.

Further, for purposes of the present invention, the term “inflammatory disease(s)” includes “autoimmune disease(s).” As used herein, the term “autoimmunity” is generally understood to encompass inflammatory immune-mediated processes involving “self” antigens. In autoimmune diseases, self antigen(s) trigger host immune responses.

Also, the present invention includes treatment of inflammation associated with tissue transplant rejection. “Transplant rejection” or “graft rejection” refers to any host-mounted immune response against a graft including but not limited to HLA antigens, blood group antigens, and the like.

The invention can also be used to treat graft versus host disease, such as that associated with bone marrow transplantation, for example. In such graft versus host disease, the donor bone marrow includes lymphocytes and cells that mature into lymphocytes. The donor's lymphocytes recognize the recipient's antigens as non-self and mount an inflammatory immune response. Hence, as used herein, “graft versus host disease” or “graft versus host reaction” refers to any T cell mediated immune response in which donor lymphocytes react to the host's antigens.

Thus, the antagonist anti-CD40 antibodies and antigen-binding fragments thereof described herein can be used in accordance with the methods of the invention to treat autoimmune and/or inflammatory disorders including, but not limited to, systemic lupus erythematosus (SLE), discoid lupus, lupus nephritis, sarcoidosis, inflammatory arthritis, including juvenile arthritis, rheumatoid arthritis, psoriatic arthritis, Reiter's syndrome, ankylosing spondylitis, and gouty arthritis, rejection of an organ or tissue transplant, hyperacute, acute, or chronic rejection and/or graft versus host disease, multiple sclerosis, hyper IgE syndrome, polyarteritis nodosa, primary biliary cirrhosis, inflammatory bowel disease, Crohn's disease, celiac's disease (gluten-sensitive enteropathy), autoimmune hepatitis, pernicious anemia, autoimmune hemolytic anemia, psoriasis, scleroderma, myasthenia gravis, autoimmune thrombocytopenic purpura, autoimmune thyroiditis, Grave's disease, Hasimoto's thyroiditis, immune complex disease, chronic fatigue immune dysfunction syndrome (CFIDS), polymyositis and dermatomyositis, cryoglobulinemia, thrombolysis, cardiomyopathy, pemphigus vulgaris, pulmonary interstitial fibrosis, Type I and Type II diabetes mellitus, type 1, 2, 3, and 4 delayed-type hypersensitivity, allergy or allergic disorders, unwanted/unintended immune responses to therapeutic proteins (see for example, U.S. Patent Application No. US 2002/0119151 and Koren, et al. (2002) Curr. Pharm. Bioteclnol. 3:349-60), asthma, Churg-Strauss syndrome (allergic granulomatosis), atopic dermatitis, allergic and irritant contact dermatitis, urtecaria, IgE-mediated allergy, atherosclerosis, vasculitis, idiopathic inflammatory myopathies, hemolytic disease, Alzheimer's disease, chronic inflammatory demyelinating polyneuropathy, and the like. In some other embodiments, the antagonistic anti-CD40 antibodies of the invention are useful in treating pulmonary inflammation including but not limited to lung graft rejection, asthma, sarcoidosis, emphysema, cystic fibrosis, idiopathic pulmonary fibrosis, chronic bronchitis, allergic rhinitis and allergic diseases of the lung such as hypersensitivity pneumonitis, eosinophilic pneumonia, bronchiolitis obliterans due to bone marrow and/or lung transplantation or other causes, graft atherosclerosis/graft phlebosclerosis, as well as pulmonary fibrosis resulting from collagen, vascular, and autoimmune diseases such as rheumatoid arthritis and lupus erythematosus.

By “anti-inflammatory activity” is intended a reduction or prevention of inflammation. Therapy with at least one anti-CD40 antibody or antigen-binding fragment thereof as defined elsewhere herein causes a physiological response that is beneficial with respect to treatment of an autoimmune disease and/or inflammatory disease, where the disease involves cells expressing the CD40 antigen. It is recognized that the methods of the invention may be useful in preventing phenotypic change in cells such as proliferation, activation, and the like.

By “positive therapeutic response” with respect to an autoimmune disease and/or inflammatory disease is intended an improvement in the disease in association with the anti-inflammatory activity of these antibodies or antigen-binding fragments thereof, and/or an improvement in the symptoms associated with the disease. That is, an anti-proliferative effect, the prevention of further proliferation of the CD40-expressing cell, a reduction in the inflammatory response including but not limited to reduced secretion of inflammatory cytokines, adhesion molecules, proteases, immunoglobulins (in instances where the CD40 bearing cell is a B cell), combinations thereof, and the like, increased production of anti-inflammatory proteins, a reduction in the number of autoreactive cells, an increase in immune tolerance, inhibition of autoreactive cell survival, and/or a decrease in one or more symptoms mediated by stimulation of CD40-expressing cells can be observed. Such positive therapeutic responses are not limited to the route of administration and may comprise administration to the donor, the donor tissue (such as for example organ perfusion), the host, any combination thereof, and the like.

For subjects undergoing therapy for an autoimmune and/or inflammatory disease, clinical response can be assessed using screening techniques such as magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like. In addition to these positive therapeutic responses, the subject undergoing therapy with the antagonist anti-CD40 antibody or antigen-binding fragment thereof may experience the beneficial effect of an improvement in the symptoms associated with the disease.

By “therapeutically effective dose or amount” is intended an amount of antagonist anti-CD40 antibody or antigen-binding fragment thereof that, when administered, brings about a positive therapeutic response with respect to treatment of a subject with a CD40-associated disease. In some embodiments of the invention, a therapeutically effective dose of the antagonist anti-CD40 antibody or fragment thereof is in the range from about 0.003 mg/kg to about 50 mg/kg, from about 0.01 mg/kg to about 40 mg/kg, from about 0.01 mg/kg to about 30 mg/kg, from about 0.1 mg/kg to about 30 mg/kg, from about 1 mg/kg to about 30 mg/kg, from about 3 mg/kg to about 30 mg/kg, from about 3 mg/kg to about 25 mg/kg, from about 3 mg/kg to about 20 mg/kg, from about 5 mg/kg to about 15 mg/kg, or from about 7 mg/kg to about 12 mg/kg. It is recognized that the method of treatment may comprise a single administration of a therapeutically effective dose or multiple administrations of a therapeutically effective dose of the antagonist anti-CD40 antibody or antigen-binding fragment thereof.

A further embodiment of the invention is the use of antagonist anti-CD40 antibodies for diagnostic monitoring of protein levels in tissue as part of a clinical testing procedure, e.g., to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

The antagonist anti-CD40 antibodies can be used in combination with known chemotherapeutics, cytokines, and/or other monoclonal antibodies, including other antagonist anti-CD40 antibodies having a different mode of action, for the treatment of CD40-associated diseases. For example, the antagonist anti-CD40 antibodies of the invention can be used in combination with cytokines such as interleukin-2. In another embodiment, the anti-CD40 antibodies of the invention can be used in combination with, for example, other monoclonal antibodies, such as rituximab (IDEC-C2B8; Rituxan®; IDEC Pharmaceuticals Corp., San Diego, Calif.) for treatment of a B cell lymphoma. In yet other embodiments, the anti-CD40 antibodies of the invention can be used in combination with anti-CD40 monoclonal antibodies that block CD40L-mediated CD40 signaling. Such a combination would potentially be useful for treating autoimmune diseases and/or inflammatory diseases, including, but not limited to, organ and tissue transplant rejection. Where the subject is undergoing transplantation of a tissue or organ, the antagonist anti-CD40 antibodies can be used in combination with other therapeutic agents that inhibit rejection of the transplanted tissue/organ. Such therapeutic agents include, but are not limited to, corticosteroids, cyclosporine, and azathioprine. Where multiple therapeutic agents are used in combination, the individual agents can be administered sequentially, in either order, or simultaneously (i.e., concurrently or within the same time frame).

In this manner, where a subject is being treated for a B cell-related cancer, including, but not limited to, those disclosed herein above, the antagonist anti-CD40 antibodies described herein, or antigen-binding fragments thereof, are administered in combination with at least one other cancer therapy, including, but not limited to, surgery or surgical procedures (e.g. splenectomy, hepatectomy, lymphadenectomy, leukophoresis, bone marrow transplantation, and the like); radiation therapy; chemotherapy, optionally in combination with autologous bone marrow transplant, where suitable chemotherapeutic agents include, but are not limited to, fludarabine or fludarabine phosphate, chlorambucil, vincristine, pentostatin, 2-chlorodeoxyadenosine (cladribine), cyclophosphamide, doxorubicin, prednisone, and combinations thereof, for example, anthracycline-containing regimens such as CAP (cyclophosphamide, doxorubicin plus prednisone), CHOP (cyclophosphamide, vincristine, prednisone plus doxorubicin), VAD (vincritsine, doxorubicin, plus dexamethasone), MP (melphalan plus prednisone), and other cytotoxic and/or therapeutic agents used in chemotherapy such as mitoxantrone, daunorubicin, idarubicin, asparaginase, and antimetabolites, including, but not limited to, cytarabine, methotrexate, 5-fluorouracil decarbazine, 6-thioguanine, 6-mercaptopurine, and nelarabine; other anti-cancer monoclonal antibody therapy (for example, alemtuzumab (Campath®) or other anti-CD52 antibody targeting the CD52 cell-surface glycoprotein on malignant B cells; rituximab (Rituxan®), the fully human antibody HuMax-CD20, R-1594, IMMU-106, TRU-015, AME-133, tositumomab/1-131 tositumomab (Bexxar®), ibritumomab tiuxetan (Zevalin®), or any other therapeutic anti-CD20 antibody targeting the CD20 antigen on malignant B cells; anti-CD19 antibody (for example, MT103, a bispecific antibody); anti-CD22 antibody (for example, the humanized monoclonal antibody epratuzumab); bevacizumab (Avastin®) or other anti-cancer antibody targeting human vascular endothelial growth factor; anti-CD22 antibody targeting the CD22 antigen on malignant B cells (for example, the monoclonal antibody BL-22, an alphaCD22 toxin); α-M-CSF antibody targeting macrophage colony stimulating factor; antibodies targeting the receptor activator of nuclear factor-kappaB (RANK) and its ligand (RANKL), which are overexpressed in multiple myeloma; anti-CD23 antibody targeting the CD23 antigen on malignant B cells (for example, IDEC-152); anti-CD80 antibody targeting the CD80 antigen (for example, IDEC-114); anti-CD38 antibody targeting the CD38 antigen on malignant B cells; antibodies targeting major histocompatibility complex class II receptors (anti-MHC antibodies) expressed on malignant B cells; other anti-CD40 antibodies targeting the CD40 antigen on malignant B cells (for example, SGN-40; and other antagonist anti-CD40 antibodies, such as CHIR-12.12 and CHIR-5.9, and antigen-binding fragments thereof, that block CD40L-mediated CD40 signaling on CD40-expressing cells, as disclosed in International Patent Application No. PCT/US2004/037152 (Attorney Docket No. PP20107.004 (035784/282916)), also entitled “Antagonist Anti-CD40 Monoclonal Antibodies and Methods for Their Use,” filed Nov. 4, 2004)); and antibodies targeting tumor necrosis factor-related apoptosis-inducing ligand receptor 1 (TRAIL-R1) (for example, the agonistic human monoclonal antibody HGS-ETR1) and TRAIL-R expressed on a number of solid tumors and tumors of hematopoietic origin); small molecule-based cancer therapy, including, but not limited to, microtubule and/or topoisomerase inhibitors (for example, the mitotic inhibitor dolastatin and dolastatin analogues; the tubulin-binding agent T900607; XL119; and the topoisomerase inhibitor aminocamptothecin), SDX-105 (bendamustine hydrochloride), ixabepilone (an epothilone analog, also referred to as BMS-247550), protein kinase C inhibitors, for example, midostaurin ((PKC-412, CGP 41251, N-benzoylstaurosporine), pixantrone, eloxatin (an antineoplastic agent), ganite (gallium nitrate), Thalomid® (thalidomide), immunomodulatory derivatives of thalidomide (for example, revlimid (formerly revimid)), Affinitak™ (antisense inhibitor of protein kinase C-alpha), SDX-101 (R-etodolac, inducing apoptosis of malignant lymphocytes), second-generation purine nucleoside analogs such as clofarabine, inhibitors of production of the protein Bcl-2 by cancer cells (for example, the antisense agents oblimersen and Genasense®), proteasome inhibitors (for example, Velcade™ (bortezomib)), small molecule kinase inhibitors (for example, CHIR-258), small molecule VEGF inhibitors (for example, ZD-6474), small molecule inhibitors of heat shock protein (HSP) 90 (for example, 17-AAG), small molecule inhibitors of histone deacetylases (for example, hybrid/polar cytodifferentiation HPC) agents such as suberanilohydroxamic acid (SAHA), and FR-901228) and apoptotic agents such as Trisenox® (arsenic trioxide) and Xcytrin® (motexafin gadolinium); vaccine/immunotherapy-based cancer therapies, including, but not limited to, vaccine approaches (for example, Id-KLH, oncophage, vitalethine), personalized immunotherapy or active idiotype immunotherapy (for example, MyVax® Personalized immunotherapy, formally designated GTOP-99), Promune® (CpG 7909, a synthetic agonist for toll-like receptor 9 (TLR9)), interferon-alpha therapy, interleukin-2 (IL-2) therapy, IL-12 therapy, IL-15 therapy, and IL-21 therapy; steroid therapy; or other cancer therapy; where the additional cancer therapy is administered prior to, during, or subsequent to the antagonist anti-CD40 antibody therapy.

Where a subject is being treated for a solid tumor comprising CD40-expressing neoplastic cells, including, but not limited to, the solid tumors disclosed herein above, the antagonist anti-CD40 antibodies described herein, or antigen-binding fragments thereof, can be administered in combination with at least one other cancer therapy, including, but not limited to, surgery, radiation therapy, chemotherapy, cytokine therapy, or other monoclonal antibody intended for use in treatment of the solid tumor of interest, where the additional cancer therapy is administered prior to, during, or subsequent to the anti-CD40 antibody therapy.

Thus, where the combined therapies comprise administration of an antagonist anti-CD40 antibody or antigen-binding fragment thereof in combination with administration of another therapeutic agent, as with chemotherapy, radiation therapy, other anti-cancer antibody therapy, small molecule-based cancer therapy, or vaccine/immunotherapy-based cancer therapy, the methods of the invention encompass coadministration, using separate formulations or a single pharmaceutical formulation, or and consecutive administration in either order. Where the methods of the present invention comprise combined therapeutic regimens, these therapies can be given simultaneously, i.e., the antagonist anti-CD40 antibody or antigen-binding fragment thereof is administered concurrently or within the same time frame as the other cancer therapy (i.e., the therapies are going on concurrently, but the antagonist anti-CD40 antibody or antigen-binding fragment thereof is not administered precisely at the same time as the other cancer therapy). Alternatively, the antagonist anti-CD40 antibody of the present invention or antigen-binding fragment thereof may also be administered prior to or subsequent to the other cancer therapy. Sequential administration of the different cancer therapies may be performed regardless of whether the treated subject responds to the first course of therapy to decrease the possibility of remission or relapse. Where the combined therapies comprise administration of the antagonist anti-CD40 antibody or antigen-binding fragment thereof in combination with administration of a cytotoxic agent, preferably the antagonist anti-CD40 antibody or antigen-binding fragment thereof is administered prior to administering the cytotoxic agent.

In some embodiments of the invention, the subject has a B-cell related cancer and the antagonist anti-CD40 antibodies described herein, or antigen-binding fragments thereof, are administered in combination with chemotherapy, and optionally in combination with autologous bone marrow transplantation, wherein the antibody and the chemotherapeutic agent(s) may be administered sequentially, in either order, or simultaneously (i.e., concurrently or within the same time frame). Examples of suitable chemotherapeutic agents include, but are not limited to, fludarabine or fludarabine phosphate, chlorambucil, vincristine, pentostatin, 2-chlorodeoxyadenosine (cladribine), cyclophosphamide, doxorubicin, prednisone, and combinations thereof, for example, anthracycline-containing regimens such as CAP (cyclophosphamide, doxorubicin plus prednisone), CHOP (cyclophosphamide, vincristine, prednisone plus doxorubicin), VAD (vincritsine, doxorubicin, plus dexamethasone), MP (melphalan plus prednisone), and other cytotoxic and/or therapeutic agents used in chemotherapy such as mitoxantrone, daunorubicin, idarubicin, asparaginase, and antimetabolites, including, but not limited to, cytarabine, methotrexate, 5-fluorouracil decarbazine, 6-thioguanine, 6-mercaptopurine, and nelarabine. In some embodiments, the antagonist anti-CD40 antibody disclosed herein, or an antigen-binding fragment thereof, is administered prior to treatment with the chemotherapeutic agent. In alternative embodiments, the antagonist anti-CD40 antibody or antigen-binding fragment thereof is administered after treatment with the chemotherapeutic agent. In yet other embodiments, the chemotherapeutic agent is administered simultaneously with the antagonist anti-CD40 antibody or antigen-binding fragment thereof.

Thus, for example, in some embodiments, the antagonist anti-CD40 antibody or antigen-binding fragment thereof is administered to a subject with a B cell-related cancer in combination with fludarabine or fludarabine phosphate. In one such embodiment, the antagonist anti-CD40 antibody or antigen-binding fragment thereof is administered prior to administration of fludarabine or fludarabine phosphate. In alternative embodiments, the antagonist anti-CD40 antibody or antigen-binding fragment thereof is administered after treatment with fludarabine or fludarabine phosphate. In yet other embodiments, the fludarabine or fludarabine phosphate is administered simultaneously with the antagonist anti-CD40 antibody or antigen-binding fragment thereof.

In other embodiments of the invention, chlorambucil, an alkylating drug, is administered to a subject with a B cell-related cancer in combination with an antagonist anti-CD40 antibody described herein or an antigen-binding fragment thereof. In one such embodiment, the antagonist anti-CD40 antibody or antigen-binding fragment thereof is administered prior to administration of chlorambucil. In alternative embodiments, the antagonist anti-CD40 antibody or antigen-binding fragment thereof is administered after treatment with chlorambucil. In yet other embodiments, the chlorambucil is administered simultaneously with the antagonist anti-CD40 antibody or antigen-binding fragment thereof.

In yet other embodiments, anthracycline-containing regimens such as CAP (cyclophosphamide, doxorubicin plus prednisone) and CHOP (cyclophosphamide, vincristine, prednisone plus doxorubicin) may be combined with administration of an antagonist anti-CD40 antibody described herein or antigen-binding fragment thereof. In one such embodiment, the antagonist anti-CD40 antibody or antigen-binding fragment thereof is administered to a subject with a B cell-related cancer prior to administration of anthracycline-containing regimens. In other embodiments, the antagonist anti-CD40 antibody or antigen-binding fragment thereof is administered to the subject after treatment with anthracycline-containing regimens. In yet other embodiments, the anthracycline-containing regimen is administered to the subject simultaneously with the antagonist anti-CD40 antibody or antigen-binding fragment thereof.

In alternative embodiments, an antagonist anti-CD40 antibody described herein or an antigen-binding fragment thereof is administered to a subject with a B cell-related cancer in combination with alemtuzumab (Campath®; distributed by Berlex Laboratories, Richmond, Calif.). Alemtuzumab is a recombinant humanized monoclonal antibody (Campath-1H) that targets the CD52 antigen expressed on malignant B cells. In one such embodiment, the antagonist anti-CD40 antibody or antigen-binding fragment thereof is administered prior to administration of alemtuzumab. In other embodiments, the antagonist anti-CD40 antibody or antigen-binding fragment thereof is administered after treatment with alemtuzumab. In yet other embodiments, the alemtuzumab is administered simultaneously with the antagonist anti-CD40 antibody or antigen-binding fragment thereof.

In alternative embodiments, an antagonist anti-CD40 antibody described herein or antigen-binding fragment thereof is administered to a subject with a B cell-related cancer in combination with a therapeutic anti-CD20 antibody targeting the CD20 antigen on malignant B cells, for example, rituximab (Rituxan®), the fully human antibody HuMax-CD20, R-1594, IMMU-106, TRU-015, AME-133, tositumomab/I-131 tositumomab (Bexxar®), or ibritumomab tiuxetan (Zevalin®). In one such embodiment, the antagonist anti-CD40 antibody or antigen-binding fragment thereof is administered to the subject prior to administration of the anti-CD20 antibody. In other embodiments, the antagonist anti-CD40 antibody or antigen-binding fragment thereof is administered to the subject after treatment with the anti-CD20 antibody. In yet other embodiments, the anti-CD20 antibody is administered to the subject simultaneously with the antagonist anti-CD40 antibody or antigen-binding fragment thereof.

Other examples of monoclonal antibodies intended for treatment of B cell-related cancers that can be used in combination with the anti-CD40 antibodies of the present invention include, but are not limited to, other antagonist anti-CD40 antibodies that block CD40L-mediated CD40 signaling, including, for example, the fully human monoclonal antibodies CHIR-12.12 and CHIR-5.9, as disclosed in International Patent Application No. PCT/US2004/037152 (Attorney Docket No. PP20107.004 (035784/282916)), also entitled “Antagonist Anti-CD40 Monoclonal Antibodies and Methods for Their Use,” filed Nov. 4, 2004)).

In alternative embodiments, an antagonist anti-CD40 antibody described herein or antigen-binding fragment thereof is administered to a subject with a B cell-related cancer in combination with a small molecule-based cancer therapy, including, but not limited to, microtubule and/or topoisomerase inhibitors (for example, the mitotic inhibitor dolastatin and dolastatin analogues; the tubulin-binding agent T900607; XL119; and the topoisomerase inhibitor aminocamptothecin), SDX-105 (bendamustine hydrochloride), ixabepilone (an epothilone analog, also referred to as BMS-247550), protein kinase C inhibitors, for example, midostaurin ((PKC-412, CGP 41251, N-benzoylstaurosporine), pixantrone, eloxatin (an antineoplastic agent), ganite (gallium nitrate), Thalomid® (thalidomide), immunomodulatory derivatives of thalidomide (for example, revlimid (formerly revimid)), Affinitak™ (antisense inhibitor of protein kinase C-alpha), SDX-101 (R-etodolac, inducing apoptosis of malignant lymphocytes), second-generation purine nucleoside analogs such as clofarabine, inhibitors of production of the protein Bcl-2 by cancer cells (for example, the antisense agents oblimersen and Genasense®), proteasome inhibitors (for example, Velcadem (bortezomib)), small molecule kinase inhibitors (for example, CHIR-258), small molecule VEGF inhibitors (for example, ZD-6474), small molecule inhibitors of heat shock protein (HSP) 90 (for example, 17-AAG), small molecule inhibitors of histone deacetylases (for example, hybrid/polar cytodifferentiation HPC) agents such as suberanilohydroxamic acid (SAHA), and FR-901228) and apoptotic agents such as Trisenox® (arsenic trioxide) and Xcytrin® (motexafin gadolinium). In one such embodiment, the antagonist anti-CD40 antibody or antigen-binding fragment thereof is administered to the subject prior to administration of the small molecule-based cancer therapy. In other embodiments, the antagonist anti-CD40 antibody or antigen-binding fragment thereof is administered to the subject after treatment with the small molecule-based cancer therapy. In yet other embodiments, the small molecule-based cancer therapy is administered to the subject simultaneously with the antagonist anti-CD40 antibody or antigen-binding fragment thereof.

In yet other embodiments, an antagonist anti-CD40 antibody described herein or an antigen-binding fragment thereof can be administered to a subject with a B cell-related cancer in combination with vaccine/immunotherapy-based cancer therapy, including, but not limited to, vaccine approaches (for example, Id-KLH, oncophage, vitalethine), personalized immunotherapy or active idiotype immunotherapy (for example, MyVax® Personalized Immunotherapy, formally designated GTOP-99), Promune® (CpG 7909, a synthetic agonist for toll-like receptor 9 (TLR9)), interferon-alpha therapy, interleukin-2 (IL-2) therapy, IL-12 therapy, IL-15 therapy, or IL-21 therapy; or steroid therapy. In one such embodiment, the antagonist anti-CD40 antibody or antigen-binding fragment thereof is administered to the subject prior to administration of the vaccine/immunotherapy-based cancer therapy. In other embodiments, the antagonist anti-CD40 antibody or antigen-binding fragment thereof is administered to the subject after treatment with the vaccine/immunotherapy-based cancer therapy. In yet other embodiments, the vaccine/immunotherapy-based cancer therapy is administered to the subject simultaneously with the antagonist anti-CD40 antibody or antigen-binding fragment thereof.

In one such embodiment, an antagonist anti-CD40 antibody described herein or an antigen-binding fragment thereof can be used in combination with 1L-2. IL-2, an agent known to expand the number of natural killer (NK) effector cells in treated patients, can be administered prior to, or concomitantly with, the antagonist anti-CD40 antibody of the invention or antigen-binding fragment thereof. Where the antagonist anti-CD40 antibody of the invention, or antigen-binding fragment thereof, has antibody-dependent cell-mediated cytotoxicity (ADCC) as another mode of action, the expanded number of effector cells with IL-2 administration may lead to enhanced ADCC activity of the administered antagonist anti-CD40 antibody or antigen-binding fragment thereof. In other embodiments, IL-21 serves as the immunotherapeutic agent to stimulate NK cell activity when administered in combination with an antagonist anti-CD40 antibody described herein or an antigen-binding fragment thereof.

In some embodiments of the invention, the subject has a solid tumor comprising CD40-expressing neoplastic cells, and the anti-CD40 antibodies described herein, or antigen-binding fragments thereof, are administered to this subject in combination with chemotherapy or cytokine therapy, wherein the antibody and the chemotherapeutic agent(s) or cytokine(s) may be administered sequentially, in either order, or simultaneously (i.e., concurrently or within the same time frame). Examples of suitable chemotherapeutic agents for subjects having a solid tumor comprising CD40-expressing neoplastic cells include, but are not limited to, CPT-11 (Irinotecan), which can be used, for example, in treating colorectal cancer and non-small cell lung cancer; gemcitabine, which can be used, for example, in treating lung cancer, breast cancer, and epithelial ovarian cancer; and other chemotherapeutic agents suitable for treatment of solid tumors. Cytokines of interest include, but are not limited to, alpha interferon, gamma interferon, interleukin-2 (IL-2), IL-12, IL-15, and IL-21, granulocyte macrophage colony stimulating factor (GM-CSF), granulocyte colony stimulating factor (G-CSF), or biologically active variants of these cytokines.

In other embodiments of the invention, the anti-CD40 antibodies described herein, or antigen-binding fragments thereof, are administered to a subject with a solid tumor comprising CD40-expressing neoplastic cells in combination with other monoclonal antibodies intended for treatment of the solid tumor. Thus, for example, where the subject is undergoing treatment for a breast cancer comprising CD40-expressing carcinoma cells, therapy could include administration of effective amounts of an antagonist anti-CD40 antibody described herein, or antigen-binding fragment thereof, in combination with administration of effective amounts of Herceptin® (Genentech, Inc., San Francisco, Calif.), which targets the Her2 receptor protein on Her2+ breast cancer cells. Similarly, where the subject is undergoing treatment for colorectal cancer comprising CD40-expressing carcinoma cells, therapy could include administration of effective amounts of an antagonist anti-CD40 antibody described herein, or antigen-binding fragment thereof, in combination with administration of effective amounts of the humanized monoclonal antibody Avastin™ (also known as bevacizumab; Genentech, Inc., San Francisco, Calif.), which binds to and inhibits vascular endothelial growth factor (VEGF), a protein that plays a critical role in tumor angiogenesis. Other examples of monoclonal antibodies intended for treatment of solid tumors that can be used in combination with the anti-CD40 antibodies of the present invention include, but are not limited to, anti-EGFR antibody targeting the epidermal growth factor receptor (for example, IMC-C225 (ImClone Systems, New York, N.Y.) (see, for example, Mendelsohn and Baselga (2000) Oncogene 19:6550-6565 and Solbach et al. (2002) Int. J. Cancer 101:390-394); anti-IGF-1 receptor antibody, targeting the IGF-1 receptor protein (see, for example, Maloney et al. (2003) Cancer Res. 63:5073-5083 and Hailey et al. (2002) Mol. Cancer. Ther. 1:1349-1353; anti-MUC1 antibody, targeting the tumor-associated antigen MUC1; anti-α5β1, anti-αvβ5, and anti-αvβ3, targeting these respective integrins, which regulate cell adhesion and signaling processes involved in cell proliferation and survival (see, for example, Laidler et al. (2000) Acta Biochimica Polonica 47(4):1159-1170 and Cruet-Hennequart et al. (2003) Oncogene 22(11):1688-1702); anti-P-cadherin antibody, targeting this cadherin family member (see, for example, copending U.S. Patent Application 20030194406); anti-VE-cadherin antibody, targeting angiogenic-related function of this endothelial cell-specific adhesion molecule (see, for example, Liao et al. (2002) Cancer Res. 62:2567-2575); and other antagonist anti-CD40 antibodies that block CD40L-mediated CD40 signaling, including, for example, the fully human monoclonal antibodies CHIR-12.12 and CHIR-5.9, as disclosed in International Patent Application No. PCT/US2004/037152 (Attorney Docket No. PP20107.004 (035784/282916)), also entitled “Antagonist Anti-CD40 Monoclonal Antibodies and Methods for Their Use,” filed Nov. 4, 2004)).

Combination therapies are also contemplated for subjects having a CD40-associated disease that comprises an autoimmune and/or inflammatory component. In this manner, where a subject is being treated for an autoimmune and/or inflammatory disease, including but not limited to the diseases disclosed herein, the antagonist anti-CD40 antibodies of the invention that target C4BP-mediated CD40 signaling, or antigen-binding fragments thereof, can be administered in combination with any known therapies for autoimmune and inflammatory diseases, including any agent or combination of agents that are known to be useful, or which have been used or are currently in use, for treatment of autoimmune and inflammatory diseases. Such therapies and therapeutic agents include, but are not limited to, surgery or surgical procedures (e.g. splenectomy, lymphadenectomy, thyroidectomy, plasmaphoresis, leukophoresis, cell, tissue, or organ transplantation, intestinal procedures, organ perfusion, and the like), radiation therapy, therapy such as steroid therapy and non-steroidal therapy, hormone therapy, cytokine therapy, therapy with dermatological agents (for example, topical agents used to treat skin conditions such as allergies, contact dermatitis, and psoriasis), immunosuppressive therapy, and other anti-inflammatory monoclonal antibody therapy, and the like. In this manner, the antagonist anti-CD40 antibodies described herein, or antigen-binding fragments thereof, are administered in combination with at least one other therapy, including, but not limited to, surgery, organ perfusion, radiation therapy, steroid therapy, non-steroidal therapy, antibiotic therapy, antifungal therapy, hormone therapy, cytokine therapy, therapy with dermatological agents (for example, topical agents used to treat skin conditions such as allergies, contact dermatitis, and psoriasis), immunosuppressive therapy, other anti-inflammatory monoclonal antibody therapy, combinations thereof, and the like.

Where the methods of the present invention comprise combined therapeutic regimens for a subject having an autoimmune disease and/or inflammatory disease, these therapies can be given simultaneously, i.e., the antagonist anti-CD40 antibody or antigen-binding fragment thereof is administered concurrently or within the same time frame as the other therapy (i.e., the therapies are going on concurrently, but the anti-CD40 antibody or antigen-binding fragment thereof is not administered precisely at the same time as the other therapy). Alternatively, the antagonist anti-CD40 antibody of the present invention or antigen-binding fragment thereof may also be administered prior to or subsequent to the other therapy. Sequential administration of the different therapies may be performed regardless of whether the treated subject responds to the first course of therapy to decrease the possibility of remission or relapse.

In some embodiments of the invention, the antagonist anti-CD40 antibodies described herein, or antigen-binding fragments thereof, are administered in combination with immunosuppressive drugs or anti-inflammatory drugs, wherein the antibody and the therapeutic agent(s) may be administered sequentially, in either order, or simultaneously (i.e., concurrently or within the same time frame). Examples of suitable immunosuppressive drugs that can be administered in combination with the antagonistic anti-CD40 antibodies of the invention include, but are not limited to, methotrexate, cyclophosphamide, mizoribine, chlorambucil, cyclosporine, such as, for example, aerosolized cyclosporine (see, U.S. Patent Application Publication No. US20020006901, herein incorporated by reference in its entirety), tacrolimus (FK506; ProGraf™), mycophenolate mofetil, and azathioprine (6-mercaptopurine), sirolimus (rapamycin), deoxyspergualin, leflunomide and its malononitriloamide analogs; and immunosuppressive proteins, including, for example, anti-CTLA4 antibodies and Ig fusions, anti-B lymphocyte stimulator antibodies (e.g., LYWPHOSTAT-B™) and Ig fusions (BLyS-Ig), anti-CD80 antibodies and etanercept (Enbrel®), as well as anti-T cell antibodies such as anti-CD3 (OKT3), anti-CD4, and the like. Examples of suitable anti-inflammatory agents include, but are not limited to, corticosteroids such as, for example, clobetasol, halobetasol, hydrocortisone, triamcinolone, betamethasone, fluocinole, fluocinonide, prednisone, prednisolone, methylprednisolone; non-steroidal anti-inflammatory drugs (NSAIDs) such as, for example, sulfasalazine, medications containing mesalamine (known as 5-ASA agents), celecoxib, diclofenac, etodolac, fenprofen, flurbiprofen, ibuprofen, ketoprofen, meclofamate, meloxicam, nabumetone, naproxen, oxaprozin, piroxicam, rofecoxib, salicylates, sulindac, and tolmetin; anti-inflammatory antibodies such as adalimumab (HUMIRA®, a TNF-αantagonist) and infliximab (Remicade®, a TNF-α antagonist), and the like. In other embodiments, a subject receiving treatment for an autoimmune and/or inflammatory disease is administered the anti-CD40 antibodies of the present invention, or suitable antigen-binding fragment thereof, in combination with other antagonist anti-CD40 antibodies that target CD40L-mediated CD40 signaling on CD40-expressing cells, for example, the antagonist anti-CD40 antibodies CHIR-12.12 and CHIR-5.9, and antigen-binding fragments thereof, as disclosed in International Patent Application No. PCT/US2004/037152 (Attorney Docket No. PP20107.004 (035784/282916)), also entitled “Antagonist Anti-CD40 Monoclonal Antibodies and Methods for Their Use,” filed Nov. 4, 2004)).

Transplant rejection and graft versus host disease can be hyperacute (humoral), acute (T cell mediated), or chronic (unknown etiology), or a combination thereof. Thus, the antagonistic anti-CD40 antibodies of the invention are used in some embodiments to prevent and/or ameliorate rejection and/or symptoms associated with hyperacute, acute, and/or chronic transplant rejection of any tissue, including, but not limited to, liver, kidney, pancreas, pancreatic islet cells, small intestine, lung, heart, corneas, skin, blood vessels, bone, heterologous or autologous bone marrow, and the like. Graft tissues may be obtained from any donor and transplanted into any recipient host, and thus the transplant procedure may comprise transplanting animal tissue to humans (e.g., xenografts), transplanting tissue from one human to another human (e.g., allografts), and/or transplanting tissue from one part of a human's body to another (e.g., autografts). Treatment with the antibodies of the invention may also reduce transplantation sequelae such as fever, anorexia, hemodynamic abnormalities, leukopenia, white cell infiltration of the transplanted organ/tissue, as well as opportunistic infections.

In some embodiments, the antagonistic anti-CD40 antibodies of the invention may be used alone or in combination with immunosuppressive drugs to treat and/or prevent transplant rejection such as hyperacute, acute, and/or chronic rejection and/or graft versus host disease. Thus, in some embodiments where the antagonistic anti-CD40 antibodies of the invention are used to treat graft rejection, the antibodies may used in combination with suitable immunosuppressive drugs, including, but not limited, to methotrexate; cyclophosphamide; mizoribine; chlorambucil; cyclosporine, such as, for example, aerosolized cyclosporine (see, U.S. Patent Application Publication No. US20020006901, herein incorporated by reference in its entirety), tacrolimus (FK506; ProGraf™), mycophenolate mofetil, and azathioprine (6-mercaptopurine), sirolimus (rapamycin), deoxyspergualin, leflunomide and its malononitriloamide analogs; immunosuppressive proteins, including, for example, anti-CTLA antibodies and Ig fusions, anti-B lymphocyte stimulator antibodies (e.g., LYMPHOSTAT-B™) and Ig fusions (BLyS-Ig), anti-CD80 antibodies and etanercept (Enbrel®), as well as anti-T cell antibodies such as anti-CD3 (OKT3), anti-CD4, and the like; or other antagonist anti-CD40 antibodies that target CD40L-mediated CD40 signaling on CD40-expressing cells, for example, the CHIR-12.12 or CHIR-5.9 antibody or antigen-binding fragment thereof.

As such, it is specifically contemplated that the compositions and methods of the invention are used in combination with other drugs to further improve symptoms and outcomes in transplant recipients, such as those receiving lung grafts, for example. Thus, in some embodiments, the antagonistic anti-CD40 antibodies of the invention are used to treat transplant rejection (such as, for example hyperacute, acute, and/or chronic rejection or graft versus host disease in lung transplant recipients) alone or in combination with parenterally and/or non-parenterally administered cyclosporine, including for example oral cyclosporine, injectable cyclosporine, aerosolized (e.g., inhaled) cyclosporine, and combinations thereof. In some embodiments where at least a component of the therapy is aerosolized cyclosporine, the cyclosporine is delivered to the lung of the recipient by inhalation of cyclosporine in aerosol spray form using, for example, a pressurized delivery device or nebulizer. The cyclosporine may be administered in either dry powder or wet form.

In some other embodiments, the antagonistic anti-CD40 antibodies of the invention may be used alone or in combination with immunosuppressive drugs to treat and/or prevent rheumatoid arthritis. Thus in some embodiments where the antagonistic anti-CD40 antibodies of the invention are used to treat rheumatoid arthritis, the antibodies may used in combination with suitable immunosuppressive drugs, including, but not limited to, methotrexate, cyclophosphamide, mizoribine, chlorambucil, cyclosporine, tacrolimus (FK506; PROGRAFT™), mycophenolate mofetil, and azathioprine (6-mercaptopurine), sirolimus (rapamycin), deoxyspergualin, leflunomide and its malononitriloamide analogs; immunosuppressive proteins, including, for example, anti-CTLA antibodies and Ig fusions, anti-B lymphocyte stimulator antibodies (e.g., LYMPHOSTAT-B™) and Ig fusions (BLyS-Ig), anti-CD20 antibodies (e.g. RITUXAN®; the fully human antibody HuMax-CD20, R-1594, IMMU-106, TRU-015, AME-133, tositumomab/I-131, tositumomab (Bexxar®), ibritumomab tituxetan (Zevalin®); anti-CD80 antibodies, and etanercept (ENBREL®), as well as anti-T cell antibodies such as anti-CD3 (OKT3), anti-CD4, and the like; or other antagonist anti-CD40 antibodies that target CD40L-mediated CD40 signaling on CD40-expressing cells, for example, the CHIR-12.12 or CHIR-5.9 antibody or antigen-binding fragment thereof. As discussed above, treatment effectiveness may be assessed using any means and includes, but is not limited to, effectiveness as measured by clinical responses defined by the American College of Rheumatology criteria, the European League of Rheumatism criteria, or any other criteria. See for example, Felson et al. (1995) Arthritis. Rheum. 38:727-35 and van Gestel et al. (1996) Arthritis Rheum. 39:34-40.

In yet other embodiments, the antagonistic anti-CD40 antibodies of the invention may be used alone or in combination with immunosuppressive drugs to treat and/or prevent multiple sclerosis. Thus in some embodiments where the antagonistic anti-CD40 antibodies of the invention are used to treat multiple sclerosis, the antibodies may used in combination with suitable immunosuppressive drugs, including, but not limited to, methotrexate, cyclophosphamide, mizoribine, chlorambucil, cyclosporine, tacrolimus (FK506; PROGRAF™), mycophenolate mofetil, and azathioprine (6-mercaptopurine), sirolimus (rapamycin), deoxyspergualin, leflunomide and its malononitriloamide analogs; immunosuppressive proteins, including, for example, anti-CTLA antibodies and Ig fusions, anti-B lymphocyte stimulator antibodies (e.g., LYMPHOSTAT-B™) and Ig fusions (BLyS-Ig), anti-CD20 antibodies (e.g., RITUXAN®; the fully human antibody HuMax-CD20, R-1594, IMMu-106, TRU-015, AME-133, tositumomab/1-131, tositumomab (Bexxar®), ibritumomab tituxetan (Zevalin®; anti-CD80 antibodies, and etanercept (ENBREL®), as well as anti-T cell antibodies such as anti-CD3 (OKT3), anti-CD4, and the like; or other antagonist anti-CD40 antibodies that target CD40L-mediated CD40 signaling on CD40-expressing cells, for example, the CHIR-12.12 or CHIR-5.9 antibody or antigen-binding fragment thereof.

Further, combination therapy with two or more therapeutic agents and an antagonist anti-CD40 antibody described herein can also be used for treatment of disease states comprising stimulated CD40-expressing cells, for example, B cell-related cancers, solid tumors, and autoimmune and/or inflammatory disorders. Without being limiting, examples include triple combination therapy, where two chemotherapeutic agents are administered in combination with an antagonist anti-CD40 antibody described herein, and where a chemotherapeutic agent and another anti-cancer monoclonal antibody (for example, alemtuzumab, rituximab, anti-CD23 antibody, or another antagonist anti-CD40 antibody such as CHIR-12.12 or CHIR-5.9 that targets CD40L-mediated CD40 signaling) are administered in combination with an antagonist anti-CD40 antibody described herein. Examples of such combinations include, but are not limited to, combinations of fludarabine, cyclophosphamide, and the antagonist anti-CD40 antibody, of the invention, or an antigen-binding fragment thereof; combinations of fludarabine, an anti-CD20 antibody, for example, rituximab (Rituxan®; IDEC Pharmaceuticals Corp., San Diego, Calif.), and the antagonist anti-CD40 antibody of the invention or an antigen-binding fragment thereof; and combinations of fludarabine, another antagonist anti-CD40 antibody that targets CD40L-mediated CD40 signaling, for example, CHIR-12.12 or CHIR 5.9, and the antagonist anti-CD40 antibody of the invention or an antigen-binding fragment thereof that targets C4BP-mediated CD40 signaling.

The antagonist anti-CD40 antibodies described herein can further be used to provide reagents, e.g., labeled antibodies that can be used, for example, to identify cells expressing CD40. This can be very useful in determining the cell type of an unknown sample. Panels of monoclonal antibodies can be used to identify tissue by species and/or by organ type. In a similar fashion, these anti-CD40 antibodies can be used to screen tissue culture cells for contamination (i.e., screen for the presence of a mixture of CD40-expressing and non-CD40 expressing cells in a culture).

Pharmaceutical Formulations and Modes of Administration

The antagonist anti-CD40 antibodies of this invention are administered at a concentration that is therapeutically effective to prevent or treat CD40-associated diseases such as autoimmunity, hypersensitivity, inflammation, auto-antibody production, organ or tissue transplant rejection, graft versus host disease, and CD40-expressing cancers such as the B-cell lymphomas and solid tumors. To accomplish this goal, the antibodies may be formulated using a variety of acceptable excipients known in the art. Typically, the antibodies are administered by injection, either intravenously or intraperitoneally. Methods to accomplish this administration are known to those of ordinary skill in the art. It may also be possible to obtain compositions which may be topically or orally administered, or which may be capable of transmission across mucous membranes.

Intravenous administration occurs preferably by infusion over a period of about 1 to about 10 hours, more preferably over about 1 to about 8 hours, even more preferably over about 2 to about 7 hours, still more preferably over about 4 to about 6 hours, depending upon the anti-CD40 antibody being administered. The initial infusion with the pharmaceutical composition may be given over a period of about 4 to about 6 hours with subsequent infusions delivered more quickly. Subsequent infusions may be administered over a period of about 1 to about 6 hours, including, for example, about 1 to about 4 hours, about 1 to about 3 hours, or about 1 to about 2 hours.

A pharmaceutical composition of the invention is formulated to be compatible with its intended route of administration. Examples of possible routes of administration include parenteral, (e.g., intravenous (IV), intramuscular (IM), intradermal, subcutaneous (SC), or infusion), oral and pulmonary (e.g., inhalation), nasal, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

The anti-CD40 antibodies are typically provided by standard technique within a pharmaceutically acceptable buffer, for example, sterile saline, sterile buffered water, propylene glycol, combinations of the foregoing, etc. Methods for preparing parenterally administrable agents are described in Remington 's Pharmaceutical Sciences (18^{th} ed.; Mack Publishing Company, Eaton, Pa., 1990), herein incorporated by reference. See also, for example, International Publication No. WO 98/56418, which describes stabilized antibody pharmaceutical formulations suitable for use in the methods of the present invention.

The amount of at least one antagonist anti-CD40 antibody or antigen-binding fragment thereof to be administered is readily determined by one of ordinary skill in the art without undue experimentation. Factors influencing the mode of administration and the respective amount of at least one antagonist anti-CD40 antibody (or antigen-binding fragment thereof) include, but are not limited to, the particular disease undergoing therapy, the severity of the disease, the history of the disease, and the age, height, weight, health, and physical condition of the individual undergoing therapy. Similarly, the amount of antagonist anti-CD40 antibody or antigen-binding fragment thereof to be administered will be dependent upon the mode of administration and whether the subject will undergo a single dose or multiple doses of this anti-tumor agent. Generally, a higher dosage of anti-CD40 antibody or antigen-binding fragment thereof is preferred with increasing weight of the patient undergoing therapy. The dose of anti-CD40 antibody or antigen-binding fragment thereof to be administered is in the range from about 0.003 mg/kg to about 50 mg/kg, preferably in the range of 0.01 mg/kg to about 40 mg/kg. Thus, for example, the dose can be 0.01 mg/kg, 0.03 mg/kg, 0.1 mg/kg, 0.3 mg/kg, 0.5 mg/kg, 1 mg/kg, 1.5 mg/kg, 2 mg/kg, 2.5 mg/kg, 3 mg/kg, 5 mg/kg, 7 mg/kg, 10 mg/kg, 15 mg/kg, 20 mg/kg, 25 mg/kg, 30 mg/kg, 35 mg/kg, 40 mg/kg, 45 mg/kg, or 50 mg/kg.

In another embodiment of the invention, the method comprises administration of multiple doses of antagonist anti-CD40 antibody or antigen-binding fragment thereof. The method may comprise administration of 1, 2, 3, 4, 5, 6, 7, 8, 9, 10, 15, 20, 25, 30, 35, 40, or more therapeutically effective doses of a pharmaceutical composition comprising an antagonist anti-CD40 antibody or antigen-binding fragment thereof. The frequency and duration of administration of multiple doses of the pharmaceutical compositions comprising anti-CD40 antibody or antigen-binding fragment thereof is dependent upon the disease, state of the disease, and medical history of the subject undergoing treatment. Moreover, treatment of a subject with a therapeutically effective amount of an antibody can include a single treatment or, preferably, can include a series of treatments. In a preferred example, a subject is treated with antagonist anti-CD40 antibody or antigen-binding fragment thereof in the range of between about 0.1 to 20 mg/kg body weight, once per week for between about 1 to 10 weeks, preferably between about 2 to 8 weeks, more preferably between about 3 to 7 weeks, and even more preferably for about 4, 5, or 6 weeks. Treatment may occur annually to prevent relapse or upon indication of relapse. It will also be appreciated that the effective dosage of antibody or antigen-binding fragment thereof used for treatment may increase or decrease over the course of a particular treatment. Changes in dosage may result and become apparent from the results of diagnostic assays as described herein.

Thus, in one embodiment, the dosing regimen includes a first administration of a therapeutically effective dose of at least one anti-CD40 antibody or antigen-binding fragment thereof on days 1, 7, 14, and 21 of a treatment period. In another embodiment, the dosing regimen includes a first administration of a therapeutically effective dose of at least one anti-CD40 antibody or antigen-binding fragment thereof on days 1, 2, 3, 4, 5, 6, and 7 of a week in a treatment period. Further embodiments include a dosing regimen having a first administration of a therapeutically effective dose of at least one anti-CD40 antibody or antigen-binding fragment thereof on days 1, 3, 5, and 7 of a week in a treatment period; a dosing regimen including a first administration of a therapeutically effective dose of at least one anti-CD40 antibody or antigen-binding fragment thereof on days 1 and 3 of a week in a treatment period; and a preferred dosing regimen including a first administration of a therapeutically effective dose of at least one anti-CD40 antibody or antigen-binding fragment thereof on day 1 of a week in a treatment period. The treatment period may comprise 1 week, 2 weeks, 3 weeks, a month, 3 months, 6 months, or a year. Treatment periods may be subsequent or separated from each other by a day, a week, 2 weeks, a month, 3 months, 6 months, or a year.

In some embodiments, the therapeutically effective doses of antagonist anti-CD40 antibody or antigen-binding fragment thereof ranges from about 0.003 mg/kg to about 50 mg/kg, from about 0.01 mg/kg to about 40 mg/kg, from about 0.01 mg/kg to about 30 mg/kg, from about 0.1 mg/kg to about 30 mg/kg, from about 0.5 mg/kg to about 30 mg/kg, from about 1 mg/kg to about 30 mg/kg, from about 3 mg/kg to about 30 mg/kg, from about 3 mg/kg to about 25 mg/kg, from about 3 mg/kg to about 20 mg/kg, from about 5 mg/kg to about 15 mg/kg, or from about 7 mg/kg to about 12 mg/kg. Thus, for example, the dose of any one antagonist anti-CD40 antibody or antigen-binding fragment thereof can be 0.003 mg/kg, 0.01 mg/kg, 0.03 mg/kg, 0.1 mg/kg, 0.3 mg/kg, 0.5 mg/kg, 1 mg/kg, 1.5 mg/kg, 2 mg/kg, 2.5 mg/kg, 3 mg/kg, 5 mg/kg, 7 mg/kg, 10 mg/kg, 15 mg/kg, 20 mg/kg, 25 mg/kg, 30 mg/kg, 35 mg/kg, 40 mg/kg, 45 mg/kg, 50 mg/kg, or other such doses falling within the range of about 0.003 mg/kg to about 50 mg/kg. The same therapeutically effective dose of an antagonist anti-CD40 antibody or antigen-binding fragment thereof can be administered throughout each week of antibody dosing. Alternatively, different therapeutically effective doses of an antagonist anti-CD40 antibody or antigen-binding fragment thereof can be used over the course of a treatment period.

In other embodiments, the initial therapeutically effective dose of an antagonist anti-CD40 antibody or antigen-binding fragment thereof as defined elsewhere herein can be in the lower dosing range (i.e., about 0.003 mg/kg to about 20 mg/kg) with subsequent doses falling within the higher dosing range (i.e., from about 20 mg/kg to about 50 mg/kg).

In alternative embodiments, the initial therapeutically effective dose of an antagonist anti-CD40 antibody or antigen-binding fragment thereof as defined elsewhere herein can be in the upper dosing range (i.e., about 20 mg/kg to about 50 mg/kg) with subsequent doses falling within the lower dosing range (i.e., 0.003 mg/kg to about 20 mg/kg). Thus, in one embodiment, the initial therapeutically effective dose of the antagonist anti-CD40 antibody or antigen-binding fragment thereof is about 20 mg/kg to about 35 mg/kg, including about 20 mg/kg, about 25 mg/kg, about 30 mg/kg, and about 35 mg/kg, and subsequent therapeutically effective doses of the antagonist anti-CD40 antibody or antigen binding fragment thereof are about 5 mg/kg to about 15 mg/kg, including about 5 mg/kg, 8 mg/kg, 10 mg/kg, 12 mg/kg, and about 15 mg/kg.

In some embodiments of the invention, antagonist anti-CD40 antibody therapy is initiated by administering a “loading dose” of the antibody or antigen-binding fragment thereof to the subject in need of antagonist anti-CD40 antibody therapy. By “loading dose” is intended an initial dose of the antagonist anti-CD40 antibody or antigen-binding fragment thereof that is administered to the subject, where the dose of the antibody or antigen-binding fragment thereof administered falls within the higher dosing range (i.e., from about 20 mg/kg to about 50 mg/kg). The “loading dose” can be administered as a single administration, for example, a single infusion where the antibody or antigen-binding fragment thereof is administered IV, or as multiple administrations, for example, multiple infusions where the antibody or antigen-binding fragment thereof is administered IV, so long as the complete “loading dose” is administered within about a 24-hour period. Following administration of the “loading dose,” the subject is then administered one or more additional therapeutically effective doses of the antagonist anti-CD40 antibody or antigen-binding fragment thereof. Subsequent therapeutically effective doses can be administered, for example, according to a weekly dosing schedule, or once every two weeks, once every three weeks, or once every four weeks. In such embodiments, the subsequent therapeutically effective doses generally fall within the lower dosing range (i.e., 0.003 mg/kg to about 20 mg/kg).

Alternatively, in some embodiments, following the “loading dose,” the subsequent therapeutically effective doses of the antagonist anti-CD40 antibody or antigen-binding fragment thereof are administered according to a “maintenance schedule,” wherein the therapeutically effective dose of the antibody or antigen-binding fragment thereof is administered once a month, once every 6 weeks, once every two months, once every 10 weeks, once every three months, once every 14 weeks, once every four months, once every 18 weeks, once every five months, once every 22 weeks, once every six months, once every 7 months, once every 8 months, once every 9 months, once every 10 months, once every 11 months, or once every 12 months. In such embodiments, the therapeutically effective doses of the antagonist anti-CD40 antibody or antigen-binding fragment thereof fall within the lower dosing range (i.e., 0.003 mg/kg to about 20 mg/kg), particularly when the subsequent doses are administered at more frequent intervals, for example, once every two weeks to once every month, or within the higher dosing range (i.e., from about 20 mg/kg to about 50 mg/kg), particularly when the subsequent doses are administered at less frequent intervals, for example, where subsequent doses are administered about one month to about 12 months apart.

The antagonist anti-CD40 antibodies present in the pharmaceutical compositions described herein for use in the methods of the invention may be native or obtained by recombinant techniques, and may be from any source, including mammalian sources such as, e.g., mouse, rat, rabbit, primate, pig, and human. Preferably such polypeptides are derived from a human source, and more preferably are recombinant, human proteins from hybridoma cell lines.

The pharmaceutical compositions useful in the methods of the invention may comprise biologically active variants of the antagonist anti-CD40 antibodies of the invention. Such variants should retain the desired biological activity of the reference antagonist anti-CD40 antibody such that the pharmaceutical composition comprising the variant antibody has the same therapeutic effect as the pharmaceutical composition comprising the reference antagonist anti-CD40 antibody when administered to a subject. That is, the variant anti-CD40 antibody will serve as a therapeutically active component in the pharmaceutical composition in a manner similar to that observed for the reference antagonist anti-CD40 antibody. Methods are available in the art for determining whether a variant anti-CD40 antibody retains the desired biological activity, and hence serves as a therapeutically active component in the pharmaceutical composition. Biological activity of antibody variants can be measured using assays specifically designed for measuring activity of the reference antagonist anti-CD40 antibody, including assays described in the present invention.

Any pharmaceutical composition comprising an antagonist anti-CD40 antibody or antigen-binding fragment thereof having the binding properties described herein as the therapeutically active component can be used in the methods of the invention. Thus liquid, lyophilized, or spray-dried compositions comprising one or more of the antagonist anti-CD40 antibodies of the invention, or antigen-binding fragment thereof, may be prepared as an aqueous or nonaqueous solution or suspension for subsequent administration to a subject in accordance with the methods of the invention. Each of these compositions will comprise at least one of the antagonist anti-CD40 antibodies of the present invention, or an antigen-binding fragment thereof, as a therapeutically or prophylactically active component. By “therapeutically or prophylactically active component” is intended the anti-CD40 antibody or antigen-binding fragment thereof is specifically incorporated into the composition to bring about a desired therapeutic or prophylactic response with regard to treatment, prevention, or diagnosis of a disease or condition within a subject when the pharmaceutical composition is administered to that subject. Preferably the pharmaceutical compositions comprise appropriate stabilizing agents, bulking agents, or both to minimize problems associated with loss of protein stability and biological activity during preparation and storage.

Formulants may be added to pharmaceutical compositions comprising an antagonist anti-CD40 antibody of the invention or antigen-binding fragment thereof. These formulants may include, but are not limited to, oils, polymers, vitamins, carbohydrates, amine acids, salts, buffers, albumin, surfactants, or bulking agents. Preferably carbohydrates include sugar or sugar alcohols such as mono-, di-, or polysaccharides, or water soluble glucans. The saccharides or glucans can include fructose, glucose, mannose, sorbose, xylose, maltose, sucrose, dextran, pullulan, dextrin, α, β, and γ cyclodextrin, soluble starch, hydroxyethyl starch, and carboxymethylcellulose, or mixtures thereof. “Sugar alcohol” is defined as a C₄ to C₈ hydrocarbon having a hydroxyl group and includes galactitol, inositol, mannitol, xylitol, sorbitol, glycerol, and arabitol. These sugars or sugar alcohols may be used individually or in combination. The sugar or sugar alcohol concentration is between 1.0% and 7% w/v., more preferably between 2.0% and 6.0% w/v. Preferably amino acids include levorotary (L) forms of carnitine, arginine, and betaine; however, other amino acids may be added. Preferred polymers include polyvinylpyrrolidone (PVP) with an average molecular weight between 2,000 and 3,000, or polyethylene glycol (PEG) with an average molecular weight between 3,000 and 5,000. Surfactants that can be added to the formulation are shown in EP Nos. 270,799 and 268,110.

Additionally, antibodies can be chemically modified by covalent conjugation to a polymer to increase their circulating half-life, for example. Preferred polymers, and methods to attach them to peptides, are shown in U.S. Pat. Nos. 4,766,106; 4,179,337; 4,495,285; and 4,609,546; which are all hereby incorporated by reference in their entireties. Preferred polymers are polyoxyethylated polyols and polyethylene glycol (PEG). PEG is soluble in water at room temperature and has the general formula: R(O—CH₂—CH₂)ₙO—R where R can be hydrogen, or a protective group such as an alkyl or alkanol group. Preferably, the protective group has between 1 and 8 carbons, more preferably it is methyl. The symbol n is a positive integer, preferably between 1 and 1,000, more preferably between 2 and 500. The PEG has a preferred average molecular weight between 1,000 and 40,000, more preferably between 2,000 and 20,000, most preferably between 3,000 and 12,000. Preferably, PEG has at least one hydroxy group, more preferably it is a terminal hydroxy group. It is this hydroxy group which is preferably activated to react with a free amino group on the inhibitor. However, it will be understood that the type and amount of the reactive groups may be varied to achieve a covalently conjugated PEG/antibody of the present invention.

Water-soluble polyoxyethylated polyols are also useful in the present invention. They include polyoxyethylated sorbitol, polyoxyethylated glucose, polyoxyethylated glycerol (POG), and the like. POG is preferred. One reason is because the glycerol backbone of polyoxyethylated glycerol is the same backbone occurring naturally in, for example, animals and humans in mono-, di-, triglycerides. Therefore, this branching would not necessarily be seen as a foreign agent in the body. The POG has a preferred molecular weight in the same range as PEG. The structure for POG is shown in Knauf et al. (1988) J. Bio. Chem. 263:15064-15070, and a discussion of POG/IL-2 conjugates is found in U.S. Pat. No. 4,766,106, both of which are hereby incorporated by reference in their entireties.

Another drug delivery system for increasing circulatory half-life is the liposome. Methods of preparing liposome delivery systems are discussed in Gabizon et al. (1982) Cancer Research 42:4734; Cafiso (1981) Biochem. Biophys. Acta 649:129; and Szoka (1980) Ann. Rev. Biophys. Eng. 9:467. Other drug delivery systems are known in the art and are described in, e.g., Poznansky et al. (1980) Drug Delivery Systems (R. L. Juliano, ed., Oxford, N.Y.), pp. 253; Poznansky (1984) Pharm. Revs. 36:277.

The formulants to be incorporated into a pharmaceutical composition should provide for the stability of the antagonist anti-CD40 antibody or antigen-binding fragment thereof. That is, the antagonist anti-CD40 antibody or antigen-binding fragment thereof should retain its physical and/or chemical stability and have the desired biological activity, i.e., one or more of the antagonist activities defined herein above, including, but not limited to, inhibition of immunoglobulin secretion by normal human peripheral B cells stimulated by T cells; inhibition of survival and/or proliferation of normal human peripheral B cells stimulated by Jurkat T cells; inhibition of survival and/or proliferation of normal human peripheral B cells stimulated by C4BP-expressing cells or soluble C4BP; inhibition of “survival” anti-apoptotic intracellular signals in any cell stimulated by soluble C4BP or solid-phase C4BP; inhibition of CD40 signal transduction in any cell upon ligation with soluble C4BP or solid-phase C4BP; and inhibition of proliferation of human malignant B cells as noted elsewhere herein.

Methods for monitoring protein stability are well known in the art. See, for example, Jones (1993) Adv. Drug Delivery Rev. 10:29-90; Lee, ed. (1991) Peptide and Protein Drug Delivery (Marcel Dekker, Inc., New York, N.Y.). Generally, protein stability is measured at a chosen temperature for a specified period of time. In preferred embodiments, a stable antibody pharmaceutical formulation provides for stability of the antagonist anti-CD40 antibody or antigen-binding fragment thereof when stored at room temperature (about 25° C.) for at least 1 month, at least 3 months, or at least 6 months, and/or is stable at about 2-8° C. for at least 6 months, at least 9 months, at least 12 months, at least 18 months, at least 24 months.

A protein such as an antibody, when formulated in a pharmaceutical composition, is considered to retain its physical stability at a given point in time if it shows no visual signs (i.e., discoloration or loss of clarity) or measurable signs (for example, using size-exclusion chromatography (SEC) or UV light scattering) of precipitation, aggregation, and/or denaturation in that pharmaceutical composition. With respect to chemical stability, a protein such as an antibody, when formulated in a pharmaceutical composition, is considered to retain its chemical stability at a given point in time if measurements of chemical stability are indicative that the protein (i.e., antibody) retains the biological activity of interest in that pharmaceutical composition. Methods for monitoring changes in chemical stability are well known in the art and include, but are not limited to, methods to detect chemically altered forms of the protein such as result from clipping, using, for example, SDS-PAGE, SEC, and/or matrix-assisted laser desorption ionization/time of flight mass spectrometry; and degradation associated with changes in molecular charge (for example, associated with deamidation), using, for example, ion-exchange chromatography. See, for example, the methods disclosed in International Patent Application No. PCT/US2004/037152 (Attorney Docket No. PP20107.004 (035784/282916)), also entitled “Antagonist Anti-CD40 Monoclonal Antibodies and Methods for Their Use,” filed Nov. 4, 2004; herein incorporated by reference in its entirety.

An antagonist anti-CD40 antibody or antigen-binding fragment thereof, when formulated in a pharmaceutical composition, is considered to retain a desired biological activity at a given point in time if the desired biological activity at that time is within about 30%, preferably within about 20% of the desired biological activity exhibited at the time the pharmaceutical composition was prepared as determined in a suitable assay for the desired biological activity. Assays for measuring the desired biological activity of the antagonist anti-CD40 antibodies disclosed herein, and antigen-binding fragments thereof, can be performed as described in the Examples herein. See also the assays described in Schultze et al. (1998) Proc. Natl. Acad. Sci. USA 92:8200-8204; Denton et al. (1998) Pediatr. Transplant. 2:6-15; Evans et al. (2000) J. Immunol. 164:688-697; Noelle (1998) Agents Actions Suppl. 49:17-22; Lederman et al. (1996) Curr. Opin. Hematol. 3:77-86; Coligan et al. (1991) Current Protocols in Immunology 13:12; Kwekkeboom et al. (1993) Immunology 79:439-444; and U.S. Pat. Nos. 5,674,492 and 5,847,082; herein incorporated by reference.

Where the antagonist anti-CD40 antibody is formulated as a liquid formulation, the liquid pharmaceutical composition is preferably lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) that may include additional ingredients. Upon reconstitution, the composition is preferably administered to subjects using those methods that are known to those skilled in the art.

Use of Antagonist Anti-CD40 Antibodies in the Manufacture of Medicaments

The present invention also provides for the use of an antagonist anti-CD40 antibody of the invention that blocks C4BP-mediated CD40 signaling, or antigen-binding fragment thereof, in the manufacture of a medicament for treating a subject for a cancer comprising CD40-expressing neoplastic cells, wherein the medicament is coordinated with treatment with at least one other cancer therapy. In some embodiments, the cancer is characterized by neoplastic B cell growth. Such cancers include, but are not limited to, the B cell-related cancers discussed herein above, for example, non-Hodgkin's lymphoma, chronic lymphocytic leukemia, multiple myeloma, B cell lymphoma, high-grade B cell lymphoma, intermediate-grade B cell lymphoma, low-grade B cell lymphoma, B cell acute lymphoblastic leukemia, myeloblastic leukemia, Hodgkin's disease, plasmacytoma, follicular lymphoma, follicular small cleaved lymphoma, follicular large cell lymphoma, follicular mixed small cleaved lymphoma, diffuse small cleaved cell lymphoma, diffuse small lymphocytic lymphoma, prolymphocytic leukemia (PLL), lymphoplasmacytic lymphoma, marginal zone lymphoma, mucosal associated lymphoid tissue lymphoma, monocytoid B cell lymphoma, splenic lymphoma, hairy cell leukemia, diffuse large cell lymphoma, mediastinal large B cell lymphoma, lymphomatoid granulomatosis, intravascular lymphomatosis, diffuse mixed cell lymphoma, diffuse large cell lymphoma, immunoblastic lymphoma, Burkitt's lymphoma, AIDS-related lymphoma, and mantle cell lymphoma. In other embodiments, the cancer is a solid tumor. Examples of solid tumors comprising CD40-expressing neoplastic cells include, but are not limited to, ovarian, lung (for example, non-small cell lung cancer of the squamous cell carcinoma, adenocarcinoma, and large cell carcinoma types, and small cell lung cancer), breast, colon, kidney (including, for example, renal cell carcinomas), bladder, liver (including, for example, hepatocellular carcinomas), gastric, cervical, prostate, nasopharyngeal, thyroid (for example, thyroid papillary carcinoma), and skin cancers such as melanoma, and sarcomas (including, for example, osteosarcomas and Ewing's sarcomas).

By “coordinated” in the context of a subject in need of treatment for a cancer is intended the medicament comprising the antagonist anti-CD40 antibody or antigen-binding fragment thereof is to be used either prior to, during, or after treatment of the subject with at least one other cancer therapy. Examples of other cancer therapies for subjects having a B cell-related cancer include, but are not limited to, surgery; radiation therapy; chemotherapy, optionally in combination with autologous bone marrow transplant, where suitable chemotherapeutic agents include, but are not limited to, fludarabine or fludarabine phosphate, chlorambucil, vincristine, pentostatin, 2-chlorodeoxyadenosine (cladribine), cyclophosphamide, doxorubicin, prednisone, and combinations thereof, for example, anthracycline-containing regimens such as CAP (cyclophosphamide, doxorubicin plus prednisone), CHOP (cyclophosphamide, vincristine, prednisone plus doxorubicin), VAD (vincritsine, doxorubicin, plus dexamethasone), MP (melphalan plus prednisone), and other cytotoxic and/or therapeutic agents used in chemotherapy such as mitoxantrone, daunorubicin, idarubicin, asparaginase, and antimetabolites, including, but not limited to, cytarabine, methotrexate, 5-fluorouracil decarbazine, 6-thioguanine, 6-mercaptopurine, and nelarabine; other anti-cancer monoclonal antibody therapy (for example, alemtuzumab (Campath®) or other anti-CD52 antibody targeting the CD52 cell-surface glycoprotein on malignant B cells; rituximab (Rituxan®), the fully human antibody HuMax-CD20, R-1594, IMMU-106, TRU-015, AME-133, tositumomab/1-131 tositumomab (Bexxar®), ibritumomab tiuxetan (Zevalin®), or any other therapeutic anti-CD20 antibody targeting the CD20 antigen on malignant B cells; anti-CD19 antibody (for example, MT103, a bispecific antibody); anti-CD22 antibody (for example, the humanized monoclonal antibody epratuzumab); bevacizumab (Avastin®) or other anti-cancer antibody targeting human vascular endothelial growth factor; anti-CD22 antibody targeting the CD22 antigen on malignant B cells (for example, the monoclonal antibody BL-22, an alphaCD22 toxin); α-M-CSF antibody targeting macrophage colony stimulating factor; antibodies targeting the receptor activator of nuclear factor-kappaB (RANK) and its ligand (RANKL), which are overexpressed in multiple myeloma; anti-CD23 antibody targeting the CD23 antigen on malignant B cells (for example, IDEC-152); anti-CD38 antibody targeting the CD38 antigen on malignant B cells; antibodies targeting major histocompatibility complex class II receptors (anti-MHC antibodies) expressed on malignant B cells; other anti-CD40 antibodies targeting the CD40 antigen on malignant B cells (for example, SGN-40; and other antagonist anti-CD40 antibodies, such as CHIR-12.12 and CHIR-5.9, and antigen-binding fragments thereof, that block CD40L-mediated CD40 signaling on CD40-expressing cells, as disclosed in International Patent Application No. PCT/US2004/037152 (Attorney Docket No. PP20107.004 (035784/282916)), also entitled “Antagonist Anti-CD40 Monoclonal Antibodies and Methods for Their Use,” filed Nov. 4, 2004)); and antibodies targeting tumor necrosis factor-related apoptosis-inducing ligand receptor 1 (TRAIL-R1) (for example, the agonistic human monoclonal antibody HGS-ETR1) expressed on a number of solid tumors and tumors of hematopoietic origin); small molecule-based cancer therapy, including, but not limited to, microtubule and/or topoisomerase inhibitors (for example, the mitotic inhibitor dolastatin and dolastatin analogues; the tubulin-binding agent T900607; XL119; and the topoisomerase inhibitor aminocamptothecin), SDX-105 (bendamustine hydrochloride), ixabepilone (an epothilone analog, also referred to as BMS-247550), protein kinase C inhibitors, for example, midostaurin ((PKC-412, CGP 41251, N-benzoylstaurosporine), pixantrone, eloxatin (an antineoplastic agent), ganite (gallium nitrate), Thalomid® (thalidomide), immunomodulatory derivatives of thalidomide (for example, revlimid (formerly revimid)), Affinitak™ (antisense inhibitor of protein kinase C-alpha), SDX-101 (R-etodolac, inducing apoptosis of malignant lymphocytes), second-generation purine nucleoside analogs such as clofarabine, inhibitors of production of the protein Bcl-2 by cancer cells (for example, the antisense agents oblimersen and Genasense®), proteasome inhibitors (for example, Velcade™ (bortezomib)), small molecule kinase inhibitors (for example, CHIR-258), small molecule VEGF inhibitors (for example, ZD-6474), small molecule inhibitors of heat shock protein (HSP) 90 (for example, 17-AAG), small molecule inhibitors of histone deacetylases (for example, hybrid/polar cytodifferentiation HPC) agents such as suberanilohydroxamic acid (SAHA), and FR-901228) and apoptotic agents such as Trisenox® (arsenic trioxide) and Xcytrin® (motexafin gadolinium); vaccine/immunotherapy-based cancer therapies, including, but not limited to, vaccine approaches (for example, Id-KLH, oncophage, vitalethine), personalized immunotherapy or active idiotype immunotherapy (for example, MyVax® Personalized Immunotherapy, formally designated GTOP-99), Promune® (CpG 7909, a synthetic agonist for toll-like receptor 9 (TLR9)), interferon-alpha therapy, interleukin-2 (IL-2) therapy, IL-12 therapy; IL-15 therapy, and IL-21 therapy; steroid therapy; or other cancer therapy; where treatment with the additional cancer therapy, or additional cancer therapies, occurs prior to, during, or subsequent to treatment of the subject with the medicament comprising the antagonist anti-CD40 antibody or antigen-binding fragment thereof, as noted herein above.

Examples of other cancer therapies for subjects having a cancer that is a solid tumor comprising CD40-expressing neoplastic cells include, but are not limited to, surgery; radiation therapy; chemotherapy, where suitable chemotherapeutic agents include, but are not limited to, fludarabine or fludarabine phosphate, chlorambucil, vincristine, pentostatin, 2-chlorodeoxyadenosine (cladribine), cyclophosphamide, doxorubicin, prednisone, and combinations thereof, for example, anthracycline-containing regimens such as CAP (cyclophosphamide, doxorubicin plus prednisone), CHOP (cyclophosphamide, vincristine, prednisone plus doxorubicin), VAD (vincritsine, doxorubicin, plus dexamethasone), MP (melphalan plus prednisone), and other cytotoxic and/or therapeutic agents used in chemotherapy such as mitoxantrone, daunorubicin, idarubicin, asparaginase, and antimetabolites, including, but not limited to, cytarabine, methotrexate, 5-fluorouracil decarbazine, 6-thioguanine, 6-mercaptopurine, and nelarabine; cytokine therapy, including, but not limited to, alpha-interferon therapy, gamma-interferon therapy, therapy with interleukin-2 (IL-2), IL-12, IL-15, and IL-21, granulocyte macrophage colony stimulating factor (GM-CSF), granulocyte colony stimulating factor (G-CSF), or biologically active variants of these cytokines; or other monoclonal antibody intended for use in treatment of the solid tumor of interest, for example, Herceptin® (Genentech, Inc., San Francisco, Calif.), which targets the Her2 receptor protein on Her2+ breast cancer cells; the humanized monoclonal antibody Avastin™ (also known as bevacizumab; Genentech, Inc., San Francisco, Calif.), which binds to and inhibits vascular endothelial growth factor (VEGF), and has use in treatment of colon cancer; anti-EGFR antibody targeting the epidermal growth factor receptor (for example, IMC-C225 (ImClone Systems, New York, N.Y.); anti-IGF-1 receptor antibody, targeting the IGF-1 receptor protein; anti-MUC1 antibody, targeting the tumor-associated antigen MUC1; anti-α5β1, anti-αvβ5, and anti-αvβ3, targeting these respective integrins, which regulate cell adhesion and signaling processes involved in cell proliferation and survival; anti-P-cadherin antibody, targeting this cadherin family member (see, for example, copending U.S. Patent Application Publication No. 20030194406); anti-VE-cadherin antibody, targeting angiogenic-related function of this endothelial cell-specific adhesion molecule; and other antagonist anti-CD40 antibodies, such as CHIR-12.12 and CHIR-5.9, and antigen-binding fragments thereof, that block CD40L-mediated CD40 signaling on CD40-expressing neoplastic cells, as disclosed in International Patent Application No. PCT/US2004/037152 (Attorney Docket No. PP20107.004 (035784/282916)), also entitled “Antagonist Anti-CD40 Monoclonal Antibodies and Methods for Their Use,” filed Nov. 4, 2004)); where treatment with the additional cancer therapy, or additional cancer therapies, occurs prior to, during, or subsequent to treatment of the subject with the medicament comprising the antagonist anti-CD40 antibody or antigen-binding fragment thereof, as noted herein above.

Thus, in some embodiments, the present invention provides for the use of the antagonist anti-CD40 antibody that blocks C4BP-mediated CD40 signaling, or antigen-binding fragment thereof, in the manufacture of a medicament for treating a B cell lymphoma, for example non-Hodgkin's lymphoma, in a subject, wherein the medicament is coordinated with treatment with at least one other cancer therapy selected from the group consisting of chemotherapy, anti-cancer antibody therapy, small molecule-based cancer therapy, and vaccine/immunotherapy-based cancer therapy, wherein the medicament is to be used either prior to, during, or after treatment of the subject with the other cancer therapy or, in the case of multiple combination therapies, either prior to, during, or after treatment of the subject with the other cancer therapies.

Thus, for example, in some embodiments, the invention provides for the use of an antagonist anti-CD40 antibody of the invention, or antigen-binding fragment thereof, in the manufacture of a medicament for treating a B cell lymphoma, for example, non-Hodgkin's lymphoma, in a subject, wherein the medicament is coordinated with treatment with chemotherapy, where the chemotherapeutic agent is selected from the group consisting of cytoxan, doxorubicin, vincristine, prednisone, and combinations thereof, for example CHOP. In other embodiments, the invention provides for the use of an antagonist anti-CD40 antibody of the invention, or antigen-binding fragment thereof, in the manufacture of a medicament for treating a B cell lymphoma, for example non-Hodgkin's lymphoma, in a subject, wherein the medicament is coordinated with treatment with at least one other anti-cancer antibody selected from the group consisting of alemtuzumab (Campath®) or other anti-CD52 antibody targeting the CD52 cell-surface glycoprotein on malignant B cells; rituximab (Rituxan®), the fully human antibody HuMax-CD20, R-1594, IMMU-106, TRU-015, AME-133, tositumomab/1-131 tositumomab (Bexxar®), ibritumomab tiuxetan (Zevalin®), or any other therapeutic anti-CD20 antibody targeting the CD20 antigen on malignant B cells; anti-CD19 antibody (for example, MT103, a bispecific antibody); anti-CD22 antibody (for example, the humanized monoclonal antibody epratuzumab); bevacizumab (Avastin®) or other anti-cancer antibody targeting human vascular endothelial growth factor; the fully human monoclonal antibody CHIR-12.12 or CHIR-5.9, or other antagonist anti-CD40 antibody that blocks CD40L-mediated CD40 signaling; and any combinations thereof; wherein the medicament is to be used either prior to, during, or after treatment of the subject with the other cancer therapy or, in the case of multiple combination therapies, either prior to, during, or after treatment of the subject with the other cancer therapies.

In yet other embodiments, the present invention provides for the use of an antagonist anti-CD40 antibody of the invention, or antigen-binding fragment thereof, in the manufacture of a medicament for treating a B cell lymphoma, for example non-Hodgkin's lymphoma, in a subject, wherein the medicament is coordinated with treatment with at least one other small molecule-based cancer therapy selected from the group consisting of microtubule and/or topoisomerase inhibitors (for example, the mitotic inhibitor dolastatin and dolastatin analogues; the tubulin-binding agent T900607; XL119; and the topoisomerase inhibitor aminocamptothecin), SDX-105 (bendamustine hydrochloride), ixabepilone (an epothilone analog, also referred to as BMS-247550), protein kinase C inhibitors, for example, midostaurin ((PKC-412, CGP 41251, N-benzoylstaurosporine), pixantrone, eloxatin (an antineoplastic agent), ganite (gallium nitrate), Thalomid™ (thalidomide), an apoptotic agent such as Xcytrin® (motexafin gadolinium), inhibitors of production of the protein Bcl-2 by cancer cells (for example, the antisense agents oblimersen and Genasense®), nelarabine, and any combinations thereof; wherein the medicament is to be used either prior to, during, or after treatment of the subject with the other cancer therapy or, in the case of multiple combination therapies, either prior to, during, or after treatment of the subject with the other cancer therapies.

In still other embodiments, the present invention provides for the use of an antagonist anti-CD40 antibody, or antigen-binding fragment thereof, in the manufacture of a medicament for treating a B cell lymphoma, for example non-Hodgkin's lymphoma, in a subject, wherein the medicament is coordinated with treatment with at least one other vaccine/immunotherapy-based cancer therapy selected from the group consisting of vaccine approaches (for example, Id-KLH, oncophage, vitalethine), personalized immunotherapy or active idiotype immunotherapy (for example, MyVax® Personalized Immunotherapy, formally designated GTOP-99), Promune® (CpG 7909, a synthetic agonist for toll-like receptor 9 (TLR9)), interleukin-2 (IL-2) therapy, IL-12 therapy; IL-15 therapy, and IL-21 therapy, and any combinations thereof; wherein the medicament is to be used either prior to, during, or after treatment of the subject with the other cancer therapy or, in the case of multiple combination therapies, either prior to, during, or after treatment of the subject with the other cancer therapies.

In some embodiments, the present invention provides for the use of the antagonist anti-CD40 antibody of the invention, or antigen-binding fragment thereof, in the manufacture of a medicament for treating a B cell-related leukemia, for example B-cell acute lymphocytic leukemia (B-ALL), in a subject, wherein the medicament is coordinated with treatment with at least one other cancer therapy selected from the group consisting of chemotherapy and anti-metabolite therapy, wherein the medicament is to be used either prior to, during, or after treatment of the subject with the other cancer therapy or, in the case of multiple combination therapies, either prior to, during, or after treatment of the subject with the other cancer therapies. Examples of such embodiments include, but are not limited to, those instances where the medicament comprising the antagonist anti-CD40 antibody or antigen-binding fragment thereof is coordinated with treatment with a chemotherapeutic agent or anti-metabolite selected from the group consisting of cytoxan, doxorubicin, vincristine, prednisone, cytarabine, mitoxantrone, idarubicin, asparaginase, methotrexate, 6-thioguanine, 6-mercaptopurine, and combinations thereof; wherein the medicament is to be used either prior to, during, or after treatment of the subject with the other cancer therapy or, in the case of multiple combination therapies, either prior to, during, or after treatment of the subject with the other cancer therapies. In one such example, the medicament is coordinated with treatment with cytarabine plus daunorubicin, cytarabine plus mitoxantrone, and/or cytarabine plus idarubicin; wherein the medicament is to be used either prior to, during, or after treatment of the B-ALL subject with the other cancer therapy or, in the case of multiple combination therapies, either prior to, during, or after treatment of the subject with the other cancer therapies.

In some embodiments, the invention provides for the use of an antagonist anti-CD40 antibody of the invention that block C4BP-mediated CD40 signaling, or antigen-binding fragment thereof, in the manufacture of a medicament for treating a subject for a solid tumor comprising neoplastic cells expressing CD40 antigen, wherein the medicament is coordinated with treatment with chemotherapy, where the chemotherapeutic agent is selected from the group consisting of CPT-11 (Irinotecan), which can be used, for example, in treating colorectal cancer and non-small cell lung cancer; gemcitabine, which can be used, for example, in treating lung cancer, breast cancer, and epithelial ovarian cancer; and other chemotherapeutic agents suitable for treatment of solid tumors; where treatment with the additional cancer therapy, or additional cancer therapies, occurs prior to, during, or subsequent to treatment of the subject with the medicament comprising the antagonist anti-CD40 antibody or antigen-binding fragment thereof, as noted herein above.

In other embodiments, the invention provides for the use of an antagonist anti-CD40 antibody of the invention, or antigen-binding fragment thereof, in the manufacture of a medicament for treating a subject for a solid tumor comprising neoplastic cells expressing CD40 antigen, wherein the medicament is coordinated with treatment with at least one other anti-cancer antibody selected from the group consisting of Herceptin® (Genentech, Inc., San Francisco, Calif.), which targets the Her2 receptor protein on Her2+ breast cancer cells; the humanized monoclonal antibody Avastin™ (also known as bevacizumab; Genentech, Inc., San Francisco, Calif.), which binds to and inhibits vascular endothelial growth factor (VEGF), and has use in treatment of colon cancer; anti-EGFR antibody targeting the epidermal growth factor receptor (for example, IMC-C225 (ImClone Systems, New York, N.Y.); anti-IGF-1 receptor antibody, targeting the IGF-1 receptor protein; anti-MUC1 antibody, targeting the tumor-associated antigen MUC1; anti-α5β1, anti-αvβ5, and anti-αvβ3, targeting these respective integrins, which regulate cell adhesion and signaling processes involved in cell proliferation and survival; anti-P-cadherin antibody, targeting this cadherin family member (see, for example, copending U.S. Patent Application Publication No. 20030194406); anti-VE-cadherin antibody, targeting angiogenic-related function of this endothelial cell-specific adhesion molecule; and the fully human monoclonal antibody CHIR-12.12 or CHIR-5.9, or other antagonist anti-CD40 antibody that blocks CD40L-mediated CD40 signaling; where treatment with the additional cancer therapy, or additional cancer therapies, occurs prior to, during, or subsequent to treatment of the subject with the medicament comprising the antagonist anti-CD40 antibody or antigen-binding fragment thereof, as noted herein above.

The invention also provides for the use of an antagonist anti-CD40 antibody of the invention, or antigen-binding fragment thereof, in the manufacture of a medicament for treating a subject for a cancer comprising CD40-expressing neoplastic cells, for example, a cancer characterized by neoplastic B cell growth, including the B cell-related cancers described herein above, or a solid tumor, wherein the medicament is used in a subject that has been pretreated with at least one other cancer therapy. By “pretreated” or “pretreatment” is intended the subject has received one or more other cancer therapies (i.e., been treated with at least one other cancer therapy) prior to receiving the medicament comprising the antagonist anti-CD40 antibody or antigen-binding fragment thereof. “Pretreated” or “pretreatment” includes subjects that have been treated with at least one other cancer therapy within 2 years, within 18 months, within 1 year, within 6 months, within 2 months, within 6 weeks, within 1 month, within 4 weeks, within 3 weeks, within 2 weeks, within 1 week, within 6 days, within 5 days, within 4 days, within 3 days, within 2 days, or even within 1 day prior to initiation of treatment with the medicament comprising the antagonist anti-CD40 antibody or antigen-binding fragment thereof. It is not necessary that the subject was a responder to pretreatment with the prior cancer therapy, or prior cancer therapies. Thus, the subject that receives the medicament comprising the antagonist anti-CD40 antibody or antigen-binding fragment thereof could have responded, or could have failed to respond (i.e. the cancer was refractory), to pretreatment with the prior cancer therapy, or to one or more of the prior cancer therapies where pretreatment comprised multiple cancer therapies. Examples of other cancer therapies for which a subject can have received pretreatment prior to receiving the medicament comprising the antagonist anti-CD40 antibody or antigen-binding fragment thereof include, but are not limited to, surgery; radiation therapy; chemotherapy, optionally in combination with autologous bone marrow transplant, where suitable chemotherapeutic agents include, but are not limited to, those listed herein above; other anti-cancer monoclonal antibody therapy, including, but not limited to, those anti-cancer antibodies listed herein above; small molecule-based cancer therapy, including, but not limited to, the small molecules listed herein above; vaccine/immunotherapy-based cancer therapies, including, but limited to, those listed herein above; steroid therapy; other cancer therapy; or any combination thereof.

“Treatment” in the context of coordinated use of a medicament described herein with one or more other cancer therapies is herein defined as the application or administration of the medicament or of the other cancer therapy to a subject, or application or administration of the medicament or other cancer therapy to an isolated tissue or cell line from a subject, where the subject has a cancer comprising CD40-expressing neoplastic cells, for example, a cancer characterized by neoplastic B cell growth or a solid tumor, a symptom associated with such a cancer, or a predisposition toward development of such a cancer, where the purpose is to cure, heal, alleviate, relieve, alter, remedy, ameliorate, improve, or affect the cancer, any associated symptoms of the cancer, or the predisposition toward the development of the cancer.

The present invention also provides for the use of an antagonist anti-CD40 antibody of the invention that blocks C4BP-mediated CD40 signaling, or antigen-binding fragment thereof, in the manufacture of a medicament for treating an autoimmune disease and/or inflammatory disease in a subject, wherein the medicament is coordinated with treatment with at least one other therapy. By “coordinated” in the context of a subject in need of treatment for an autoimmune disease and/or inflammatory disease is intended the medicament is to be used either prior to, during, or after treatment of the subject with at least one other therapy. Examples of other therapies for autoimmune and/or inflammatory diseases include, but are not limited to, those described herein above, i.e., surgery or surgical procedures (e.g. splenectomy, lymphadenectomy, thyroidectomy, plasmaphoresis, leukophoresis, cell, tissue, or organ transplantation, organ perfusion, intestinal procedures, and the like), radiation therapy, therapy such as steroid therapy and non-steroidal therapy, hormone therapy, cytokine therapy, therapy with dermatological agents (for example, topical agents used to treat skin conditions such as allergies, contact dermatitis, and psoriasis), immunosuppressive therapy, and other anti-inflammatory monoclonal antibody therapy, and the like, where treatment with the additional therapy, or additional therapies, occurs prior to, during, or subsequent to treatment of the subject with the medicament comprising the antagonist anti-CD40 antibody or antigen-binding fragment thereof, as noted herein above. In one such embodiment, the present invention provides for the use of an antagonist anti-CD40 antibody of the invention, or antigen-binding fragment thereof, in the manufacture of a medicament for treating an autoimmune disease and/or inflammatory disease in a subject, wherein the medicament is coordinated with treatment with at least one other therapy as noted herein above.

In some embodiments, the medicament comprising the antagonist anti-CD40 antibody of the invention or antigen-binding fragment thereof is coordinated with treatment with two other therapies. Where the medicament comprising the antagonist anti-CD40 antibody or antigen-binding fragment thereof is coordinated with two other therapies, use of the medicament can be prior to, during, or after treatment of the subject with either or both of the other therapies.

The invention also provides for the use of an antagonist anti-CD40 antibody that blocks C4BP-mediated CD40 signaling, or antigen-biding fragment thereof, in the manufacture of a medicament for treating an autoimmune disease and/or inflammatory disease in a subject, wherein the medicament is used in a subject that has been pretreated with at least one other therapy. By “pretreated” or “pretreatment” is intended the subject has been treated with one or more other therapies prior to receiving the medicament comprising the antagonist anti-CD40 antibody or antigen-binding fragment thereof. “Pretreated” or “pretreatment” includes subjects that have been treated with the other therapy, or other therapies, within 2 years, within 18 months, within 1 year, within 6 months, within 2 months, within 6 weeks, within 1 month, within 4 weeks, within 3 weeks, within 2 weeks, within 1 week, within 6 days, within 5 days, within 4 days, within 3 days, within 2 days, or even within 1 day prior to initiation of treatment with the medicament comprising the antagonist anti-CD40 antibody or antigen-binding fragment thereof. It is not necessary that the subject was a responder to pretreatment with the prior therapy, or prior therapies. Thus, the subject that receives the medicament comprising the antagonist anti-CD40 antibody or antigen-binding fragment thereof could have responded, or could have failed to respond, to pretreatment with the prior therapy, or to one or more of the prior therapies where pretreatment comprised multiple therapies.

“Treatment” in the context of coordinated use of a medicament described herein with one or more other therapies for an autoimmune disease and/or inflammatory disease is herein defined as the application or administration of the medicament or of the other therapy to a subject, or application or administration of the medicament or other therapy to an isolated tissue or cell line from a subject, where the subject has an autoimmune disease and/or inflammatory disease, a symptom associated with an autoimmune disease and/or inflammatory disease, or a predisposition toward development of an autoimmune disease and/or inflammatory disease, where the purpose is to cure, heal, alleviate, relieve, alter, remedy, ameliorate, improve, or affect the autoimmune disease and/or inflammatory disease, any associated symptoms of the autoimmune disease and/or inflammatory disease, or the predisposition toward the development of the autoimmune disease and/or inflammatory disease.

The following examples are offered by way of illustration and not by way of limitation.

### EXPERIMENTAL

The following protocols may be used in the examples described below.

ELISA Assay for Immunoglobulin Quantification

The concentrations of human IgM and IgG are estimated by ELISA. 96-well ELISA plates are coated with 2 μg/ml goat anti-human IgG mAb (The Jackson Laboratory, Bar Harbor, Me.) or with 2 μg/ml goat anti-human IgM mAb 4102 (Bio Source International, California) in 0.05 M carbonate buffer (pH 9.6), by incubation for 16 hours at 4° C. Plates are washed 3 times with PBS-0.05% Tween-20 (PBS-Tween) and saturated with BSA for 1 hour. After 2 washes the plates are incubated for 2 hours at 37° C. with different dilutions of the test samples. After 3 washes, bound Ig is detected by incubation for 2 hours at 37° C. with 1 μg/ml peroxidase-labeled goat anti-human IgG mAb or goat anti-human IgM mAb. Plates are washed 4 times, and bound peroxidase activity is revealed by the addition of O-phenylenediamine as a substrate. Human IgG or IgM standards (Caltaq, Burlingame, Calif.) are used to establish a standard curve for each assay.

Isolation of the Peripheral Blood Mononuclear Cells (PBMC) from Human Peripheral Blood

20 ml of Ficoll-Paque solution (low endotoxin; Pharmacia) is added per 50 ml polystyrene tube, in 3 tubes, 30 minutes before adding the blood. The Ficoll-Paque solution is warmed up to room temperature. 3 L of bleach in 1:10 dilution is prepared, and used to wash all the tubes and pipettes contacting the blood. The blood is layered on the top of the Ficoll-Paque solution without disturbing the Ficoll layer, at 1.5 ml blood/1 ml of Ficoll-Paque. The tubes are centrifuged at 1700 rpm for 30 minutes at room temperature with the brake on the centrifuge turned off. As much of the top layer (plasma) as possible is removed, minimizing the vacuum in order to avoid removing the second layer of solution. The second layer, which contains the B and T lymphocytes, is collected using a sterile Pasteur pipette, and placed in two 50-ml polystyrene tubes. The collection is diluted with 3× the volume of cold RPMI with no additives, and the tubes are centrifuged at 1000 RPM for 10 minutes. The media is removed by aspiration, and the cells from both 50-ml tubes are resuspended in a total of 10 ml cold RPMI (with additives) and transferred to a 15-ml tube. The cells are counted using the hemacytometer, then centrifuged at 1000 RPM for 10 minutes. The media is removed and the cells resuspended in 4 ml RPMI. This fraction contains the PBMC.

Isolation of the B cells from PBMC

100 μl of Dynabeads (anti-CD19) are placed in a 5-ml plastic tube. 3 ml of sterile PBS are added to the beads and mixed, and placed in the magnetic holder, then allowed to sit for 2 minutes. The solution is removed using a Pasteur pipette. 3 ml of sterile PBS are added, mixed, and placed in the magnetic holder, then allowed to sit for 2 minutes. This procedure with sterile PBS is repeated one more time for a total of 3 washes. The PBMC is added into the beads and incubated, while mixing, for 30 minutes at 40° C. The tube containing the PBMC and beads is placed into the magnetic holder for 2 minutes, then the solution is transferred to a new 5-ml tube in the magnetic holder. After 2 minutes, the solution is transferred to a new 15-ml tube. This step is repeated four more times, and the solutions of the first four times are collected in the 15-ml tube, then centrifuged at 1000 RPM for 5 minutes. This step produces the pellet for T-cell separation.

100 μl RPMI (with additives) is added to collect the beads, and the solution is transferred into a 0.7-ml tube. 101 of Dynal Detacha Beads are added into the suspension at room temperature, and it is allowed to rotate for 45 minutes. The suspension is transferred into a new 5-ml tube and 3-ml of RPMI (with additives) are added. The tube is placed in the magnetic holder for 2 minutes. The solution is transferred into a new 5-ml tube in the holder for 2 minutes, then to a 15-ml tube. The previous step is repeated three more times, collecting the solution in the 15-ml tube. The 15-ml tube is centrifuged at 1000 RPM for 10 minutes, and the cells resuspended in 10 ml RMPI. The washing step is repeated 2 more times for a total of 3 washes. The cells are counted before the last centrifugation. This step completes the B-cell purification. Cells are stored in 90% FCS and 10% DMSO and frozen at −80° C.

Flow Cytofluorometric Assay

Ramos cells (10⁶ cells/sample) are incubated in 100 μl primary antibody (10 μg/ml in PBS-BSA) for 20 min at 4° C. After 3 washes with PBS-BSA or HBSS-BSA, the cells are incubated in 100 μl FITC-labeled F(ab′)₂ fragments of goat anti-(human IgG) antibodies (Caltaq) for 20 min at 4° C. After 3 washes with PBS-BSA and 1 wash with PBS, the cells are resuspended in 0.5-ml PBS. Analyses are performed with a FACSCAN V (Becton Dickinson, San Jose, Calif.).

Generation of Hybridoma Clones

Splenocytes from immunized mice are fused with SP 2/0 or P 3×63Ag8.653 murine myeloma cells at a ratio of 10:1 using 50% polyethylene glycol as previously described by de Boer et al. (1988) J. Immunol. Meth. 113:143. The fused cells are resuspended in complete IMDM medium supplemented with hypoxanthine (0.1 mM), aminopterin (0.01 mM), thymidine (0.016 mM), and 0.5 ng/ml hIL-6 (Genzyme, Cambridge, Mass.). The fused cells are then distributed between the wells of 96-well tissue culture plates, so that each well contains 1 growing hybridoma on average.

After 10⁻¹⁴ days the supernatants of the hybridoma populations are screened for specific antibody production. For the screening of specific antibody production by the hybridoma clones, the supernatants from each well are pooled and tested for anti-CD40 activity specificity by ELISA first. The positives are then used for fluorescent cell staining of EBV-transformed B cells as described for the FACS assay above. Positive hybridoma cells are cloned twice by limiting dilution in IMDM/FBS containing 0.5 ng/ml hIL-6.

### Example 1

### Production of Anti-CD40 Antibodies

Transgenic mice bearing the human IgG1 or IgG2 heavy chain locus and the human κ chain locus (Abgenix γ−1 xenomouse) are used to generate anti-CD40 antibodies. SF9 insect cells expressing CD40 extracellular domain are used as immunogen. Mice spleens are fused with the mouse myeloma SP2/0 cells to generate antibodies that recognize recombinant CD40 in ELISA. On average approximately 10% of hybridomas produced in Abgenix xenomice may contain mouse lambda light chain instead of human kappa chain. The antibodies containing mouse light lambda chain are selected out. A subset of antibodies that also show binding to cell-surface CD40 is selected for further analysis. Stable hybridomas selected during a series of subcloning procedures are used for further characterization in binding and functional assays. Clones from other hybridomas are further identified as having antagonistic activity. Based on their relative antagonist potency and ability to inhibit C4BP-mediated CD40 signaling, and thus impact CD40-directed activities, hybridoma clones are selected for further evaluation.

### Example 2

### Binding Properties of Selected Hybridomas

Protein A is immobilized onto CM5 biosensor chips via amine coupling. Anti-CD40 monoclonal antibodies, at 1.5 μg/ml, are captured onto the modified biosensor surface for 1.5 minutes at 10 μl/min. Recombinant soluble CD40-his is flowed over the biosensor surface at varying concentrations. Antibody and antigen are diluted in 0.01 M HEPES pH 7.4, 0.15 M NaCl, 3 mM EDTA, 0.005% Surfactant P20 (HBS-EP). Kinetic and affinity constants are determined using the Biaevaluation software with a 1:1 interaction model/global fit.

### Example 3

### Effect of Antagonist Anti-CD40 Antibodies on the CD40/C4BP Interaction In vitro

In some instances, the candidate antibodies will prevent the binding of C4BP to cell surface CD40 and displace the pre-bound C4BP. Candidate antibodies are tested for their ability to prevent C4BP binding to CD40 on the surface of a lymphoma cell line (Ramos). Binding of suitable antagonist anti-CD40 antibodies to CD40 antigen on these cells prevents the subsequent binding of PE-C4BP, FITC-C4BP, biotin-C4BP, or Alexfluor-C4BP, as measured by flow cytometric assays. In a second set of assays, the candidate antibodies are tested for their ability to displace C4BP pre-bound to cell surface CD40.

### Example 4

### Antibody Antagonists for CD40-Directed Proliferation of Human Lymphocytes from Normal Subjects

Engagement of CD40 by C4BP stimulates CD40 signaling, which induces proliferation of normal human B cells. An antagonist anti-CD40 antibody that blocks C4BP-mediated CD40 signaling is expected to inhibit this proliferation.

Candidate antibodies are tested for their ability to inhibit C4BP-mediated CD40 signaling and subsequent proliferation of PBMC from normal human subjects. Soluble C4BP alpha chain subunit and C4BP α7β1 heteromer are used as agonists. The proliferation of PBMC is measured by tritiated-thymidine incorporation. The experiment is performed with multiple donors of PBMC (n>1) to ensure that the observed inhibition is not a peculiarity of cells from a single donor. A wide range of antibody concentrations (0.01 μg/ml to 100 μg/ml) is used in these assays. Generally, antibodies of interest will interfere with C4BP-mediated CD40 signaling, thereby inhibiting CD40-directed proliferation, at 0.1 μg/ml concentration of antibodies in most cases.

In addition to B cells, human PBMC also contain natural killer cells that can mediate antibody dependent cytotoxicity (ADCC). To clarify the mechanism of antibody-mediated inhibition of proliferation, assays are performed with B cells purified from human PBMC. If antibodies can inhibit CD40-directed proliferation of purified B cells that is induced by binding of C4BP to CD40, then the antagonist activity of the candidate antibodies, and not the mechanism of ADCC, causes proliferation inhibition in these assays.

### Example 5

### Antagonist Antibodies do not Induce Strong Proliferation of Human B Cells from Normal Subjects

C4BP induces normal B cells to proliferate. Binding of agonist anti-CD40 antibodies can provide a similar stimulatory signal for the proliferation of normal and malignant B cells. Antibodies with strong B cell stimulatory activity are not suitable candidates for therapeutic treatment of B cell lymphomas and autoimmune disorders. The candidate antibodies that block C4BP-mediated CD40 signaling are tested for their ability to induce proliferation of B cells from normal volunteer donors. The B cells purified from normal donor PBMC are cultured with varying concentrations of candidate antibodies (range of 0.001 to 100 μg/ml) for a total of 4 days. The B cell proliferation is measured by incorporation of tritiated thymidine. While soluble C4BP induces vigorous proliferation of B cells, candidate antibodies that are suitable for methods of the present invention induce only weak proliferation of normal B cells.

In addition to B cells, human PBMC contain cell types that bear Fc receptors (FcR) for IgG1 molecules that can provide cross linking of anti-CD40 antibodies bound to CD40 on B cells. This cross-linking could potentially enhance stimulatory activity of anti-CD40 antibodies. To confirm the lack of B cell stimulatory activity of candidate antibodies in the presence of cross-linking cells, proliferation experiments are performed with total PBMC containing B cells as well as FcR⁺ cells. Generally, these candidate antibodies even in the presence of FcR-bearing cells do not stimulate B cells to proliferate over background proliferation induced by control human IgG1. The lack of stimulatory activity by candidate mAbs is further confirmed by measuring the PBMC proliferation in response to candidate anti-CD40 antibodies immobilized on the plastic surface of culture wells. Taken together these data show that the candidate anti-CD40 antibodies do not possess strong B cell stimulatory properties.

### Example 6

### Candidate Antibodies are Able to Kill CD40-Bearing Target Cells by ADCC

In some instances, the candidate antibodies can kill CD40-bearing target cells (lymphoma lines and/or solid tumor lines) by the mechanism of ADCC. Antibodies of the IgG1 isotype are expected to have the ability to induce the killing of target cells by the mechanism of ADCC. The candidate anti-CD40 antibodies are tested for their ability to kill cancer cell lines in in vitro assays. Two human lymphoma cell lines (Ramos and Daudi), one human colon cancer cell line (HCT116), and seven other carcinoma cell lines, including the ovarian cancer cell lines SKOV3 and HEY, the skin squamous cancer cell line A431, the breast cancer cell lines MDA-MB231 and MDA-MB435, and the lung cancer cell lines NC1—H460 and SK-MES-1 are selected as target cells for these assays. PBMC or enriched NK cells from normal volunteer donors are used as effector cells in these assays.

### Example 7

### Candidate Antibodies do not Stimulate Proliferation of Cancer Cells from the Lymph Nodes of NHL Patients

CD40 signaling induces survival and proliferation of lymphoma cells from NHL patients. As such C4BP may play a role in NHL. Binding of some anti-CD40 antibodies (agonist) can provide a similar stimulatory signal for the proliferation of patient cancer cells. As noted above, antibodies with strong B cell stimulatory activity are not suitable candidates for therapeutic treatment of B cell lymphomas. Candidate antibodies are tested for their ability to induce proliferation of NHL cells from patients. The cells isolated from lymph node (LN) biopsies are cultured with varying concentrations of candidate antibodies (range of 0.01 to 300 μg/ml) for a total of 3 days. The cell proliferation is measured by incorporation of tritiated thymidine. Generally, candidate mAbs should not induce any proliferation of cancer cells at any concentration tested. Antibodies even in the presence of exogenously added IL-4, a B cell growth factor, should not induce proliferation of NHL cells. These results will indicate whether candidate antibodies are non-agonist anti-CD40 antibodies and do not stimulate proliferation in vitro of NHL cells from patients.

### Example 8

### Candidate Antibodies can Block C4BP-Induced Proliferation of Cancer Cells from Non-Hodgkin's Lymphoma Patients

Engagement of CD40 by C4BP may induce proliferation of cancer cells from NHL patients. Candidate antagonist anti-CD40 antibodies are expected to inhibit this proliferation. Candidate anti-CD40 antibodies are tested at varying concentrations (0.01 μg/ml to 100 μg/ml) for their ability to inhibit CD40-directed proliferation of NHL cells that is induced by the binding of C4BP to CD40 antigen on these cells. NHL cells from patients are cultured in suspension with C4BP in the presence of IL-4. The NHL cell proliferation is measured by ³H-thymidine incorporation. Candidate antibodies of interest inhibit the proliferation of NHL cells when compared to the control in a dose-dependent manner, as the inhibitory effect increases with increasing antagonist anti-CD40 antibody concentration.

### Example 9

### Effect of Candidate Antibodies on Number of Viable NHL Cells when Cultured with C4BP-Expressing Cells

Binding of C4BP to CD40 is an alternative method of stimulating CD40-expressing cells. CD40 signaling is important for B cell survival. This set of experiments evaluates the effect of candidate anti-CD40 antibodies on NHL cell numbers at days 7, 10, and 14 in the presence of C4BP. NHL cells from patients are cultured in suspension with C4BP in the presence of IL-4. The control human IgG and candidate antibodies are added at concentrations of 10 μg/ml at day 0 and day 7. The viable cells under each condition are counted on the specified day. Cell numbers in the control group (IgG) generally increases with time. Generally, reduced numbers of cells are recovered in the presence of antagonist antibodies as compared to control group.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims disclosed herein. Although specific terms are employed herein, they are used in a generic and descriptive sense only and not for purposes of limitation.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

1. An antibody, or antigen-binding fragment thereof, that is capable of specifically binding to a CD40 antigen expressed on the surface of a cell, said antibody or antigen-binding fragment thereof being free of significant CD40 agonist activity, wherein binding of said antibody to said CD40 antigen blocks C4b binding protein (C4BP)-mediated CD40 signaling.

2. The antibody of claim 1, wherein said antibody is a monoclonal antibody.

3. The antibody of claim 2, wherein said monoclonal antibody is selected from the group consisting of a fully human anti-CD40 monoclonal antibody, a humanized anti-CD40 monoclonal antibody, and an immunologically active chimeric anti-CD40 monoclonal antibody.

4-5. (canceled)

6. The antibody of claim 1, wherein said antibody binds to said CD40 antigen with an affinity (K_{D}) of at least about 10⁻⁶ M to about 10⁻² M.

7. The antibody of claim 1, wherein said antigen-binding fragment thereof is selected from the group consisting of a Fab fragment, an F(ab′)₂ fragment, an Fv fragment, and a single-chain Fv fragment.

8. The antibody of claim 1, wherein said antibody binds at least a portion of a C4BP binding site on CD40 with higher affinity than does C4BP.

9. The antibody of claim 1, wherein binding of said antibody or antigen-binding fragment thereof to said CD40 antigen blocks C4BP-mediated CD40 signaling by sterically inhibiting the binding of said C4BP to said CD40 antigen.

10. The antibody of claim 1, wherein said antibody or antigen-binding fragment thereof competitively inhibits binding of C4BP to said CD40 antigen by competing for at least a portion of a C4BP binding site on said CD40 antigen.

11. The antibody of claim 1, wherein binding of said antibody or antigen-binding fragment thereof to said CD40 antigen prevents CD40 signal transduction when said C4BP ligates to said CD40 antigen.

12. The antibody or antigen-binding fragment thereof according to claim 1, wherein said antibody or antigen-binding fragment thereof is recombinantly produced.

13. A hybridoma cell line capable of producing the monoclonal antibody of claim 2.

14. An antagonist anti-CD40 antibody that binds to at least a portion of a C4b binding protein (C4BP) binding site on CD40.

15. The antibody of claim 14, wherein said antibody is a fully human antibody.

16. The antibody of claim 14, wherein said antibody is free of significant CD40 agonist activity.

17. The antibody of claim 14, wherein binding of said antibody or antigen-binding fragment thereof to said CD40 antigen blocks C4BP-mediated CD40 signaling by sterically inhibiting the binding of said C4BP to said CD40 antigen.

18. The antibody of claim 14, wherein said antibody or antigen-binding fragment thereof competitively inhibits binding of C4BP to said CD40 antigen by competing for at least a portion of a C4BP binding site on said CD40 antigen.

19. The antibody of claim 14, wherein binding of said antibody or antigen-binding fragment thereof to said CD40 antigen prevents CD40 signal transduction when said C4BP ligates to said CD40 antigen.

20. The antibody of claim 14, wherein the antibody binds at least a portion of the C4BP-binding site on CD40 with higher affinity than does C4BP.

21. A method for inhibiting a CD40-directed activity of a CD40-expressing cell, where said CD40-directed activity is mediated by the binding of C4b binding protein (C4BP) to CD40 antigen expressed on the surface of said cell, said method comprising contacting said cell with an effective amount of an anti-CD40 antibody, or antigen binding fragment thereof, that is capable of specifically binding to said CD40 antigen, said anti-CD40 antibody or antigen-binding fragment thereof being free of significant CD40 agonist activity, wherein binding of said antibody to said CD40 antigen on said CD40-expressing cell blocks C4BP-mediated CD40 signaling, thereby inhibiting said CD40-directed activity.

22. The method of claim 21, wherein said CD40-directed activity is selected from the group consisting of cell proliferation, cell differentiation, antibody production, cell memory generation, isotype switching, intercellular adhesion, secretion of cytokines, secretion of metalloproteases, and expression of cell adhesion molecules.

23. The method of claim 22, wherein said CD40-expressing cell is a normal B cell, and said CD40-directed activity that is inhibited is selected from the group consisting of cell proliferation, cell differentiation, and antibody production.

24. The method of claim 22, wherein said CD40-expressing cell is a malignant B cell or a CD40-expressing neoplastic cell of a solid tumor, and said CD40-directed activity that is inhibited is cell proliferation.

25. The method of claim 21, wherein binding of said anti-CD40 antibody or antigen-binding fragment thereof to said CD40 antigen blocks C4BP-mediated CD40 signaling by sterically inhibiting the binding of said C4BP to said CD40 antigen.

26. The method of claim 21, wherein said anti-CD40 antibody or antigen-binding fragment thereof competitively inhibits binding of C4BP to said CD40 antigen by competing for at least a portion of a C4BP binding site on said CD40 antigen.

27. The method of claim 21, wherein binding of said anti-CD40 antibody or antigen-binding fragment thereof to said CD40 antigen prevents CD40 signal transduction when said C4BP ligates to said CD40 antigen.

28. The method of claim 21, wherein the anti-CD40 antibody is a monoclonal antibody.

29. The method of claim 28, wherein said monoclonal antibody is selected from the group consisting of a fully human anti-CD40 monoclonal antibody, a humanized anti-CD40 monoclonal antibody, and an immunologically active chimeric anti-CD40 monoclonal antibody.

30-31. (canceled)

32. The method of claim 21, wherein said anti-CD40 antibody binds to said CD40 antigen with an affinity (K_{D}) of at least about 10⁻⁶ M to about 10⁻¹² M.

33. The method of claim 21, wherein said antigen-binding fragment is selected from the group consisting of a Fab fragment, an F(ab′)₂ fragment, an Fv fragment, and a single-chain Fv fragment.

34. A method for treating a CD40-associated disease in a subject in need thereof, said method comprising administering to said subject a therapeutically effective amount of an antibody or antigen-binding fragment thereof, wherein said antibody or antigen-binding fragment thereof is selected from the group consisting of:
a) an antibody or antigen-binding fragment thereof, that is capable of specifically binding to a CD40 antigen expressed on the surface of a cell, said antibody or antigen-binding fragment thereof being free of significant CD40 agonist activity, wherein binding of said antibody to said CD40 antigen blocks C4b binding protein (C4BP)-mediated CD40 signaling; and
b) an antagonist anti-CD40 antibody or antigen-binding fragment thereof, that binds to at least a portion of a C4b binding protein (C4BP) binding site on CD40.

35. The method of claim 34, wherein said CD40-associated disease is a cancer.

36. The method of claim 35, wherein said cancer is a B cell-related cancer selected from the group consisting of cancer is selected from the group consisting of non-Hodgkin's lymphoma, chronic lymphocytic leukemia, multiple myeloma, B cell lymphoma, high-grade B cell lymphoma, intermediate-grade B cell lymphoma, low-grade B cell lymphoma, B cell acute lymphoblastic leukemia, myeloblastic leukemia, Hodgkin's disease, plasmacytoma, follicular lymphoma, follicular small cleaved lymphoma, follicular large cell lymphoma, follicular mixed small cleaved lymphoma, diffuse small cleaved cell lymphoma, diffuse small lymphocytic lymphoma, prolymphocytic leukemia, lymphoplamacytic lymphoma, marginal zone lymphoma, mucosal associated lymphoid tissue lymphoma, monocytoid B cell lymphoma, splenic lymphoma, hairy cell leukemia, diffuse large cell lymphoma, mediastinal large B cell lymphoma, lymphomatoid granulomatosis, intravascular lymphomatosis, diffuse mixed cell lymphoma, diffuse large cell lymphoma, immunoblastic lymphoma, Burkitt's lymphoma, AIDS-related lymphoma, and mantle cell lymphoma.

37. The method of claim 35, wherein said cancer is a solid tumor comprising neoplastic cells expressing CD40 antigen.

38. The method of claim 37, wherein said solid tumor is selected from the group consisting of lung carcinoma, breast carcinoma, ovarian carcinoma, skin carcinoma, colon carcinoma, urinary bladder carcinoma, liver carcinoma, gastric carcinoma, prostate cancer, renal cell carcinoma, nasopharyngeal carcinoma, squamous cell carcinoma, thyroid papillary carcinoma, cervical carcinoma, and sarcomas.

39. The method of claim 36, further comprising administering to said subject at least one other cancer therapy intended for use in treatment of said cancer, wherein said at least one other cancer therapy is selected from the group consisting of surgery, radiation therapy, chemotherapy, other anti-cancer monoclonal antibody therapy, small molecule-based cancer therapy, vaccine-based cancer therapy, immunotherapy-based cancer therapy, and steroid therapy.

40. The method of claim 34, wherein said CD40-associated disease is an inflammatory disease or an autoimmune disease.

41. The method of claim 40, wherein said inflammatory disease or autoimmune disease is selected from the group consisting of systemic lupus erythematosus (SLE), discoid lupus, lupus nephritis, sarcoidosis, juvenile arthritis, rheumatoid arthritis, psoriatic arthritis, Reiter's syndrome, ankylosing spondylitis, gouty arthritis, rejection of an organ or tissue transplant, graft versus host disease, multiple sclerosis, hyper IgE syndrome, polyarteritis nodosa, primary biliary cirrhosis, inflammatory bowel disease, Crohn's disease, celiac's disease (gluten-sensitive enteropathy), autoimmune hepatitis, pernicious anemia, autoimmune hemolytic anemia, psoriasis, scleroderma, myasthenia gravis, autoimmune thrombocytopenic purpura, autoimmune thyroiditis, Grave's disease, Hashimoto's thyroiditis, immune complex disease, chronic fatigue immune dysfunction syndrome (CFIDS), polymyositis and dermatomyositis, cryoglobulinemia, thrombolysis, cardiomyopathy, pemphigus vulgaris, pulmonary interstitial fibrosis, sarcoidosis, Type I and Type II diabetes mellitus, type 1, 2, 3, and 4 delayed-type hypersensitivity, allergy or allergic disorders, asthma, Churg-Strauss syndrome (allergic granulomatosis), atopic dermatitis, allergic and irritant contact dermatitis, urtecaria, IgE-mediated allergy, atherosclerosis, vasculitis, idiopathic inflammatory myopathies, hemolytic disease, Alzheimer's disease, and chronic inflammatory demyelinating polyneuropathy.

42. The method of claim 41, wherein said organ or tissue transplant is selected from the group consisting of heart, lung, kidney, pancreas, skin, and bone marrow.

43. The method of claim 41, wherein said treatment further comprises administering to said subject at least one other therapy selected from the group consisting of surgery, organ perfusion, radiation therapy, steroid therapy, non-steroidal therapy, antibiotic therapy, antifungal therapy, hormone therapy, cytokine therapy, therapy with dermatological agents, immunosuppressive therapy, and other anti-inflammatory monoclonal antibody therapy.

44. The method of claim 43, wherein said CD40-associated disease is rejection of an organ or tissue transplant, and the subject is also administered an immunosuppressive agent selected from the group consisting of cyclosporine, FK506, rapamycin, corticosteroids, CTLA4-Ig, and anti-B Lymphocyte Stimulator antibody.

45. A method for identifying an antibody that inhibits binding of C4BP to CD40 antigen, comprising performing a competitive binding assay between C4BP and an antibody that binds CD40.

46. A human monoclonal antibody that is capable of specifically binding to a human CD40 antigen expressed on the surface of a human CD40-expressing cell, said monoclonal antibody being free of significant agonist activity, wherein binding of said antibody to said CD40 antigen blocks C4BP-mediated CD40 signaling of said cell.
